(19) United States
(12) Patent Application Publication
(10) Pub. No.: US 2004/0018238 A1
(43) Pub. Date: Jan. 29, 2004

(54) Biodegradable vehicles and delivery systems of biolgically active substances

(76) Inventor: Atul J Shukla, Cordova, TN (US)

Correspondence Address: Joseph R Snyder, Townsend and Townsend and Crew, 8th Floor, Two Embarcadero Street, San Francisco, CA, 94111, US

(21) Appl. No.: 10/312,394

(22) PCT Filed: Feb. 26, 2001
(86) PCT No.: PCT/US01/06138

(51) Int. Cl. A61K 9/14

(52) U.S. Cl. 424/486

(57) ABSTRACT

Biodegradable vehicle and delivery systems of physiologically, pharmacologically and biologically active substance(s) (BAS) are provided. The biodegradable vehicles may be prepared by blending biodegradable polymers and plasticizers using a novel solvent evaporation method. This method involves dissolving the biodegradable polymer or copolymer and a plasticizer or mixtures of plasticizers into a volatile solvent or mixtures of volatile solvents. The volatile solvent is then removed using vacuum or at an elevated temperature or using a combination of both vacuum and elevated temperature. The biodegradable vehicle can be used as filler or spacer in the body. Biologically active substances (BAS) can be added to the biodegradable vehicle at any step during or after preparing the biodegradable vehicle, or just prior to using the biodegradable system. This biodegradable delivery system provides controlled release of the BAS over the desired period of time. The biodegradable vehicle or BAS-loaded biodegradable delivery system can be injected, implanted, smeared or applied in vivo in an animal, bird or human.

### CROSS-REFERENCE TO RELATED APPLICATIONS

This application claims priority to U.S. patent application Ser. No. 09/605,661, filed Jun. 28, 2000, the teachings of which are incorporated herein by reference in their entirety for all purposes.

### FIELD OF THE INVENTION

Biodegradable vehicles and delivery systems, which can be mixed with one or more physiologically, pharmacologically and biologically active substance(s) (BAS), are provided. The biodegradable vehicle (without any BAS-loading) can be used as a biodegradable filler or spacer to fill in cavities or body tissues in animals, birds and humans. The biodegradable vehicle can be mixed with one or more BAS. The delivery systems loaded with BAS can be used to control the release of the BAS from the delivery system for a prolonged period of time. The consistency and rheology, hydrophilicity and hydrophobicity, and in vivo degradation rates of the biodegradable vehicles and BAS loaded delivery systems are controlled by modulating the types of polymers or copolymers, molecular weight of polymers and copolymers, copolymer ratios, and ratios of blends of polymers or copolymers with different molecular weights or different hydrophilicity or hydrophobicity, types of plasticizers, concentration of plasticizers, ratios of two or more plasticizers used in combination. The release characteristics of the BAS from the biodegradable delivery system are also controlled by the above-mentioned factors. The present invention also provides methods for preparing these biodegradable vehicles and delivery systems.

### BACKGROUND OF THE INVENTION

The term biodegradable polymers refer to those polymers, which are slowly converted to nontoxic degradation products in the body. Examples include homopolymers and copolymers of polylactic acid or polylactide (PLA), polyglycolic acid or polyglycolide, polycaprolactone (PCL), polyanhydrides, polyphosphoesters, polyorthoesters, polyaminoacids, pseudopolyaminoacids, polyhydroxybutyrates, polyhydroxyvalerates, polyphosphazenes, polyalkylcyanoacrylates, polydioxanone, poly(ε-decaloactone), poly(glycolide-co-trimethylene carbonate), poly(ethylene carbonate), poly(iminocarbonate), poly(1,3-propylene malonate), poly(ethylene-1,4-phenylene-bis-oxyacetate), poly(ester-amides). Some of these polymers and their copolymers have been studied extensively for biomedical applications such as sutures, staples and mesh for wound closure, fracture fixation, bone augmentation and ligament reconstruction in orthopedics, ligation clips and vascular grafts in cardiovascular surgery, and dental repairs (Barrows T. Degradable implant materials: a review of synthetic absorbable polymers and their applications. Clinical materials., 1:233-257, 1986). They have also been used to prepare biodegradable drug delivery systems capable of releasing the drug or a biologically active substance over the desired length of time.

The advantages of using biodegradable polymers in biodegradable delivery systems of BAS are: ready availability of polymers, polymers used are nontoxic, biocompatibile and biodegradable, facile predictability of biodegradation rates of the polymers, ease of modification of the degradation characteristics of the polymers, regulatory approval of some of the commonly used biodegradable polymers, ease of fabrication of the polymers into various types of devices and the possibility of controlling the release of BAS by polymers over the desired length of time.

Release of BAS from a polymeric delivery system depends on the physicochemical characteristics of the BAS molecule, polymer and other excipients, and the dosage form. The important factors governing BAS release characteristics from the delivery systems prepared with biodegradable polymers are polymer molecular weight, copolymer ratio, polymer hydrophilicity or lipophilicity, percentage of various polymers in a blend consisting of polymers with varying molecular weights or copolymer ratios, hydrophilicity or hydrophilicity of the platicizer, percentage of various hydrophilic and hydrophilic plasticizers in a blend of varying types of plasticizers, degree of plasticization, particle size and percentage of BAS-loading, hydrophilicity or lipophilicity of the incorporated BAS, solubility of the BAS in both the delivery system and in the biological fluids, physical form of the formulation (i.e. liquid, gel or paste), and the method of preparation of the delivery system.

Several types of BAS delivery systems have been prepared from biodegradable polymers. These include microparticles such as microspheres and microcapsules (Schindler A, Jeffcoat R, Kimmel G L, Pitt C G, Wall M E and Zwelinger R., in: Contemporary Topics in Polymer Science, Pearce E M and Schaefgen J R, eds., Vol. 2, Plenum Publishing Corporation, New York, pp. 251-289, 1977; Mason N S, Gupta D V S, Keller, D W, Youngquist R S, and Sparks R F. Biomedical applications of microencapsulation, (Lim F, ed.), CRC Press Inc., Florida, pp. 75-84, 1984; Harrigan S E, McCarthy D A, Reuning R and Thies C., Midl. Macromol. Monograph, 5:91-100, 1978.; Sanders L M, Burns R, Bitale K and Hoffman P., Clinical performance of nafarelin controlled release injectable: influence of formulation parameters on release kinetics and duration of efficacy., Proceedings of the International Symposium on Controlled Release and Bioactive Materials, 15:62-63, 1988; Mathiowitz E, Leong K and Langer R., Macromolecular drug release from bioerodible polyanhydride microspheres, in: Proceedings of the 12th International Symposium on Controlled Release of Bioactive Materials, Peppas N and Haluska R, eds., pp. 183, 1985), films (Jackanicz T M, Nash H A, Wise D L and Gregory J B. Polylactic acid as a biodegradable carrier for contraceptive steroids., Contraception, 8:227-233, 1973.; Woodland J H R, Yolles S, Blake A B, Helrich M and Meyer F J. Long-acting delivery systems for narcotic antagonist. I. J. Med. Chem., 16:897-901, 1973), fibers (Eenink M J D, Maassen G C T, Sam A P, Geelen J A A, van Lieshout J B J M, Olijslager J, de Nijs H, and de Jager E. Development of a new long-acting contraceptive subdermal implant releasing 3-ketodesogeatrel., Proceedings of the 15th International Symposium on Controlled Release of Bioactive Materials, Controlled Release Society, Lincolnshire, Ill., pp. 402-403, 1988), capsules (Sidman K R, Schwope A D, Steber W D, Rudolph S E, Paulin S B. Biodegradable, implantable sustained release systems based on glutamic acid copolymers. J. Membr. Sci., 7:277-291, 1980; Pitt C G, Gratzl M M, Jeffcoat M A, Zweidinger R and Schindler A. Sustained drug delivery systems II: Factors affecting release rates from poly-ε-caprolactone and related biodegradable polyesters., J. Pharm. Sci., 68(12):1534-1538, 1979), discs (Cowsar D R, Dunn R L., Biodegradable and non-biodegradable fibrous delivery systems, in: Long acting Contraceptive Delivery Systems, Zatuchni G I, Goldsmith A, Shelton J D and Sciarra J J, eds., Harper & Row, Publishers, Philadelphia, pp. 145-148, 1984), wafers (Brem et al., J. Neurosurgery, 74:441-446, 1991) and solutions (Dunn et al., U.S. Pat. Nos. 4,938,763; 5,324,519; 5,324,520; 5,278,201; 5,340,849; 5,368,859; 5,660,849; 5,632,727; 5,599,552; 5,487,897). All of these, with the exception of microparticles need to be surgically implanted. This procedure is inconvenient and undesirable. Drug-loaded microspheres on the other hand, can be easily injected. However, there are several inherent disadvantages of microparticles. These include the need for reconstitution before injection, the inability to remove the dose once it is injected, and the relatively complicated manufacturing procedure.

In addition, all the drug delivery systems described in the aforementioned section contains at least one BAS, which is incorporated into the drug delivery system during the manufacturing of the dosage form. It is often difficult (if not impossible) to individualize BAS dosing (or change the BAS-loading) in these drug delivery systems. Also, there exists a possibility where a certain percentage of BAS often degrades because of its exposure to the solvents, chemicals or other harsh manufacturing conditions during the preparation of the drug delivery system or during storage of the finished product.

Therefore, there clearly exists a need for developing easily injectable, implantable, smearable or applicable biodegradable vehicles and BAS-loaded biodegradable delivery systems such as free-flowing or viscous liquids, gels, and pastes, prepared from biodegradable polymers using alternative methods. Moreover, there is also a need for developing a more versatile delivery vehicle where the type of BAS and the dose of BAS can be tailored (to individualize BAS dosing) just prior to its use. The stability of the BAS can also be enhanced in such a delivery system where the BAS is loaded into the vehicle just prior to use.

### SUMMARY OF THE INVENTION

In certain aspects, the present invention relates to compositions and methods of preparing biodegradable vehicles and delivery systems. The present invention also provides compositions of biodegradable vehicles and BAS-loaded delivery systems, and the process of blending one or more BAS with the biodegradable vehicles. The biodegradable vehicles can be used as biodegradable fillers or spacers (e.g., an artificial tissue) to fill in cavities or body tissues in animals, birds and humans. One or more biologically active substances (BAS) can be loaded into the biodegradable vehicle to prepare the biodegradable delivery system, which can be used to control the release of the BAS over a desired period of time.

In one aspect, the present invention provides a biodegradable vehicle comprising at least one biodegradable polymer having at least one plasticizer. Preferably, the plasticizer is capable of modulating the consistency, the hydrophobicity, hydrophilicity and degradation characteristics of the biodegradable vehicle. The biodegradable vehicle preferably has at least one biologically active substance mixed therewith. The biodegradable polymer or blends thereof is/are capable of modulating the degradation kinetics of the biodegradable vehicle and in certain instances, the consistency, the hydrophobicity and the hydrophilicity of the biodegradable vehicle as well. The plasticizer or blends thereof are also capable of modulating the degradation kinetics, the consistiency, the hydrophilicity and the hydrophobicity of the biodegradable vehicle as well.

In another aspect, the present invention provides a biodegradable delivery system comprising: (a) at least one biodegradable polymer, the polymer selected from polyesters, polyorthoesters, polylactides, polyglycolides, polycaprolactones, polyhydroxybutyrates, polyhydroxyvalerates, polyamides and polyanhydrides; and (b) at least two plasticizers, one of the plasticizers being hydrophilic and the other of the plasticizers being hydrophobic; and (c) at least one biologically active substance.

The method of manufacturing the biodegradable vehicles described in the present invention involves dissolving one or more biodegradable polymers and one or more plasticizers in a volatile solvent or mixture of volatile solvents. The volatile solvent or mixture of volatile solvents is/are then removed using vacuum or evaporated at an elevated temperature, or removed using both vacuum and elevated temperature. The resulting biodegradable vehicles can be free flowing or viscous liquids, gels or pastes. This method is particularly suited when polymers of high molecular weights are used to prepare the vehicles or BAS delivery system, or when a high consistency of the biodegradable vehicle or BAS delivery system, is desired. Alternatively, one or more biodegradable polymers can be directly dissolved in one or more plasticizers by stirring the mixture with or without the use of heat. This method is particularly suited when polymers of low molecular weights are used to prepare the biodegradable vehicles or BAS delivery system, or when a low consistency or BAS delivery system is desired.

In order to prepare a BAS-loaded delivery system, the BAS can be loaded into the biodegradable vehicle in any physical form (i.e. solid, liquid, gel or paste, where the BAS is dissolved or suspended in the plasticizer or mixtures of plasticizers, volatile solvents or mixture of volatile solvents or mixtures of volatile solvents and plasticizers) at any step during the manufacturing process of biodegradable delivery systems before the volatile solvent is completely removed. The BAS-loaded delivery system can also be manufactured by loading the BAS soon after the biodegradable vehicle is prepared, or blending the BAS to the biodegradable vehicle just prior to the use of the BAS-loaded biodegradable delivery system. Mixing of the BAS with the biodegradable vehicle can be accomplished by simply stirring the mixture with a stirring device, or by triturating the mixture or employing an ointment mill or a suitable device or apparatus or equipment that can be used for blending/mixing. When the BAS is blended with the biodegradable vehicle just prior to use, it could be stored in a separate container in a solid state, liquid state (where the BAS is dissolved or suspended in the plasticizer or blends of plasticizers), or gel or paste (where the BAS is dissolved or suspended in the plasticizer or blends of plasticizers). Alternatively, a device, which resembles two syringes or syringe-like devices (e.g. pumps in which materials can be mixed by depressing a trigger-like device) attached together with a removable partition or a valve assembly can also be used to uniformly mix the BAS with the biodegradable vehicle. The BAS is loaded in one syringe or compartment and the biodegradable vehicle is loaded in the other compartment. A removable partition or a valve, which will allow the contents of the two compartments to be mixed uniformly, separates the two compartments. The mixing process is performed in order to dissolve or uniformly suspend the BAS particles in the biodegradable vehicle. The resulting BAS-loaded biodegradable delivery systems can be free flowing or viscous liquids, gels or pastes. In order to prepare a BAS-loaded delivery system just prior to use, the BAS and the biodegradable vehicle can be packaged in two separate containers as a kit. The vehicle and the BAS can then be blended together by the aforementioned methods.

The biodegradable vehicles or BAS-loaded biodegradable delivery systems could be sterilized in the final package by an appropriate technique such as irradiation sterilization technique. Alternatively, the biodegradable vehicles or BAS-loaded biodegradable delivery systems can be prepared from pre-sterilized components in an aseptic environment. Sterilization of the solvents and plasticizers used in the manufacturing process could be accomplished by an appropriate sterilization technique such as filtration, autoclaving or irradiation. The polymer and the BAS used to prepare the biodegradable vehicles and the BAS-loaded biodegradable delivery systems could also be sterilized by an appropriate sterilizing technique.

Advantages of the biodegradable vehicles described in the present invention include the ease of manufacturing, injection, implantation, and application, ease of control over the consistency or rheology and hydrophilicity or hydrophobicity of the biodegradable vehicle, flexibility of tailoring in vivo degradation kinetics of the vehicles, tailoring the dose of the BAS in the biodegradable delivery systems by blending the requisite amount of BAS with the biodegradable vehicle, and enhancing stability of the BAS, especially when it is blended with the biodegradable vehicle just prior to its use. A major reason for the enhanced stability of the BAS is that the BAS is not subjected to exposure to solvents, chemicals or the harsh processing conditions especially during the manufacture of the biodegradable vehicle. Moreover, if the BAS is stored in an appropriate separate container, it does not come in contact with the biodegradable vehicle until it is blended with the vehicle.

Advantages of biodegradable delivery systems of the present invention include ease of manufacturing, injection, implantation, and application, ease of control over the consistency or rheology and hydrophilicity or hydrophobicity of the biodegradable delivery systems, ease of incorporation of BAS into the delivery systems, facile tailoring of the release of BAS from the biodegradable delivery systems, and control of in vivo biodegradation rates of biodegradable delivery systems.

The biodegradable vehicles without blending any BAS may be used as a tissue or cavity fillers or spacers in the body, whereas the biodegradable vehicles loaded with BAS may be used for the treatment of a variety of diseases and pathological conditions.

The final composition with or without the BAS may be injected, implanted, smeared or applied directly in animals, birds and humans.

In still yet another embodiment, the present invention provides a kit comprising a) a biodegradable vehicle; and b) a BAS. In certain aspects, the BAS is blended with the biodegradable vehicle just prior to use. In certain aspects, the BAS is stored in a separate container in a solid state, liquid state (where the BAS is dissolved or suspended in the plasticizer or blends of plasticizers), or gel or paste (where the BAS is dissolved or suspended in the plasticizer or blends of plasticizers). Alternatively, a device, which resembles two syringes or syringe-like devices (e.g. pumps in which materials can be mixed by depressing a trigger-like device) attached together with a removable partition or a valve assembly can also be used to uniformly mix the BAS with the biodegradable vehicle.

Further embodiments and advantages will become more apparent when read with the detailed descriptions and figures that follow.

### BRIEF DESCRIPTION OF THE DRAWINGS

FIG. 1 shows a method of preparing a biodegradable vehicle and delivery systems without the use of volatile solvents.

FIG. 2 shows a method of preparing a biodegradable vehicle and delivery systems.

FIG. 3 shows an alternate method of preparing biodegradable delivery systems.

FIG. 4 describes the effect of varying polymer to plasticizer ratios on cumulative amount of levonorgestrel released from biodegradable delivery systems.

FIG. 5 describes the effect of varying polymer inherent viscosity on cumulative amount of levonorgestrel released from biodegradable delivery systems.

FIG. 6 describes the effect of varying copolymer ratios on cumulative amount of levonorgestrel released from biodegradable delivery systems.

FIG. 7 describes the effect of varying drug loadings on oxytetracycline base released from biodegradable delivery systems.

FIG. 8 describes the effect of varying plasticizer compositions on oxytetracycline base released from biodegradable delivery systems.

FIG. 9 describes the effect of varying plasticizer to polymer ratios on oxytetracycline base released from biodegradable delivery systems.

FIG. 10 describes the effect of varying hydrophilicity of plasticizers on oxytetracycline base released from biodegradable delivery systems.

FIG. 11 describes the effect of varying polymer to plasticizer ratios and plasticizer compositions on oxytetracycline base released from biodegradable delivery systems.

FIG. 12 describes the effect of varying polymer molecular weights on oxytetracycline base released from biodegradable delivery systems.

FIG. 13 describes the effect of varying drug solubility on naltrexone released from biodegradable delivery systems.

FIG. 14 describes the effect of varying solubility of drug on oxytetracycline released from biodegradable delivery systems.

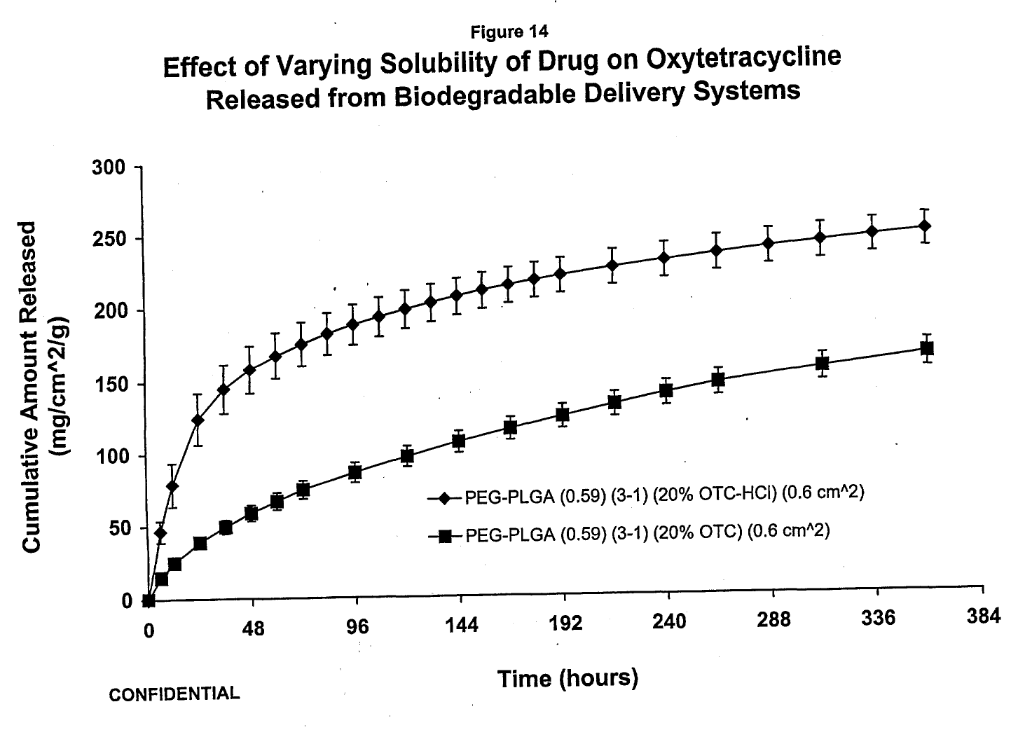

FIG. 15 describes the effect of varying polymer molecular weights on oxytetracycline base released from biodegradable delivery systems.

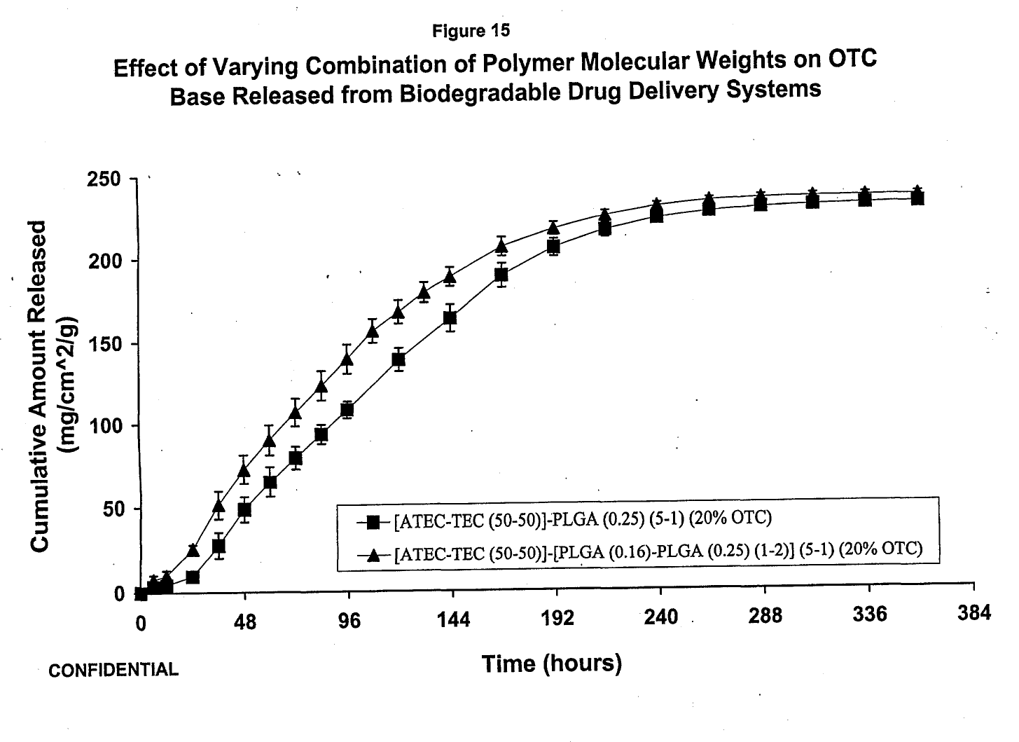

FIG. 16 describes the effect of varying polymer molecular weights on in vivo release of oxytetracycline base from biodegradable delivery systems.

### DETAILED DESCRIPTION OF THE INVENTION

In certain embodiments, the present invention relates to compositions of biodegradable vehicles and BAS-loaded delivery systems comprising at least one polymer and at least one plasticizer. The delivery system of the present invention may also comprise of at least one biologically active substance (BAS). It also relates to the method of preparing biodegradable vehicles and delivery systems loaded with BAS.

According to the present invention, the term polymer includes oligomer, homopolymer, copolymer and terpolymer. Biodegradable polymers are used in this invention because they form matrices that can control the release of BAS over a desired length of time, can degrade in vivo into non-toxic degradation products, and are available in varying physicochemical properties including varying hydrophilicity and hydrophobicity, varying molecular weights, varying crystallinity and amorphous states, and varying copolymer ratios.

In the present invention, plasticizers are used in varying ratios to convert a polymer in a solid state to a biodegradable vehicle or delivery system of varying consistency such as a free flowing or a viscous liquid, a gel or a paste. Plasticizers are chemicals added to polymers to improve their flow, and therefore their processibility (Billmeyer, F., Jr. Textbook of Polymer Science, John Wiley and Sons, New York, 1984, p. 472). This is achieved by lowering their glass transition temperature (a temperature at which a glassy polymer becomes rubbery on heating and a rubbery polymer reverts to a glassy one on cooling), thus achieving a change in properties. A plasticizer can only plasticize a polymer when the molecules of the plasticizer can interact with the molecules of the polymer. Hence, the plasticizers act like lubricants between the polymer chains, facilitating slippage of chain past chain under stress and extending the temperature range for segmental rotation to lower temperatures (Mart, A., Physical Pharmacy, Lea and Febiger, Philadelphia, 1993, p. 588). The degree or extent of plasticization of a polymer will depend on the type and amount of plasticizer blended with the polymer. For example, higher the concentration of the plasticizer, greater the extent of plasticization or flexibility of the polymer. If a plasticizer and a polymer are fully compatible with each other, then depending on the concentration of the plasticizer blended with the polymer, it is possible to obtain a polymer matrix of varying consistency or rheology such as a free-flowing or viscous liquid, gel or paste. Moreover, since plasticizers are available with varying physicochemical properties, including varying hydrophilicity and lipophilicity, it is possible to blend an appropriate plasticizer at a desired concentration with a selected compatible polymer such that the resulting biodegradable vehicle or BAS-loaded biodegradable delivery system has the tailored physicochemical characteristics, including varying hydrophilicity and lipophilicity, and consistency. The present invention also includes formulations wherein two or more plasticizers are used in a combination or blend of varying ratios. The present invention also includes formulations wherein two or more polymers or copolymers with varying copolymer ratios or molecular weights are used in a combination or blend of varying ratios.

Methods of preparing the biodegradable vehicles and delivery systems of the present invention involve dissolving at least one biodegradable polymer in a volatile solvent or a mixture of solvents. At least one plasticizer is added to the resulting polymer solution. The volatile solvent is evaporated using vacuum or removed at an elevated temperature, or evaporated using a combination of both vacuum and elevated temperature. The resulting biodegradable vehicles and delivery systems could be in the form of either free-flowing or viscous liquids, gels or pastes. This method is particularly suited when polymers of high molecular weights are used to prepare the vehicles or BAS delivery system, or when a high consistency of the biodegradable vehicle or BAS delivery system, is desired. Alternatively, one or more biodegradable polymers can be directly dissolved in one or more plasticizers by stirring the mixture with or without the use of heat. This method is particularly suited when polymers of low molecular weights are used to prepare the biodegradable vehicles or BAS delivery system, or when a low consistency or BAS delivery system is desired.

Polymers suitable for preparing the biodegradable delivery systems of the present invention include, but are not limited to, homopolymers and/or copolymers of polyesters, polyorthoesters, polyphosphoesters, polyanhydrides, polyaminoacids, pseudopolyamino acids, polyamides, polyalkylcyanoacrylates, polyphosphazenes, polydioxanone, poly(ε-decaloactone), poly(glycolide-co-trimethylene carbonate), poly(ethylene carbonate), poly(iminocarbonate), poly(1,3-propylene malonate), poly(ethylene-1,4phenylene-bis-oxyacetate), and poly(ester-amides). In a preferred embodiment, polymers include polylactic acid or polylactide (PLA) and its copolymers, polyglycolic acid or polyglycolide and its copolymers, polycaprolactone (PCL) and its copolymers, polyhydroxybutyrates and their copolymers, and polyhydroxyvalerates and polydioxanone and their copolymers. A mixture of polymers with different molecular weights or different types, or copolymer ratios may be used to tailor physicochemical properties, the degradation characteristics of the biodegradable vehicles and the delivery systems or the release characteristics of BAS from the biodegradable delivery systems, or both.

Solvents used to dissolve the polymer for the preparation of biodegradable delivery system of the present invention include, but are not limited to, ketones, ethers, alcohols, amides, and chlorinated solvents. Preferred solvents are acetone, ethyl acetate, methyl acetate, methylethylketone, chloroform, methylene chloride, isopropanol, ethyl alcohol, ethyl ether, methylethyl ether, hexafluroisopropanol, tertrahydrofuran, and hexafluroacetone sesquihydrate. A mixture of volatile solvents may also be used to create a suitable mixture, which can dissolve both the polymer and the plasticizer.

Plasticizers used for the preparation of biodegradable delivery system of the present invention include, but are not limited to, citrates such as diethyl citrate (DEC), triethyl citrate (TEC), acetyl triethyl citrate (ATEC), tributyl citrate (TBC), acetyl tributyl citrate (ATBC), butyryltri-n-hexyl-citrate, acetyltri-n-hexyl citrate, phthalates such as dimethyl phthalate (DMP), diethyl phthalate (DEP), dibutyl phthalate (DBP), dioctyl phthalate, glycol ethers such as ethylene glycol diethyl ether, propylene glycol monomethyl ether, ethylene glycol monoethyl ether, diethylene glycol monoethyl ether (Transcutol®), propylene glycol monotertiary butyl ether, dipropylene glycol monomethyl ether, N-methyl-2-pyrrolidone, 2 pyrrolidone (2-Pyrrol®), isopropyl myristate, isopropyl palmitate, dimethylacetamide, propylene glycol, glycerol, glyceryl dioleate, ethyl oleate, benzylbenzoate, glycofurol, sorbitol, sucrose acetate isobutyrate, sebacates such as dibutyl sebacate, dipropylene glycol methyl ether acetate (DPM acetate), propylene carbonate, propylene glycol laurate, propylene glycol caprylate/caprate, gamma butyrolactone, polyethylene glycols (PEG), vegetable oils obtained from seeds, flowers, fruits, leaves, stem or any part of a plant or tree such as cotton seed oil, soy bean oil, almond oil, sunflower oil, peanut oil, sesame oil, glycerol and PEG esters of acids and fatty acids (Gelucires®, Labrafils® and Labrasol®) such as PEG-6 glycerol mono oleate, PEG-6 glycerol linoleate, PEG-8 glycerol linoleate, PEG-4 glyceryl caprylate/caprate, PEG-8 glyceryl caprylate/caprate, polyglyceryl-3-oleate, polyglyceryl-6-dioleate, polyglyceryl-3-isostearate, PEG-32 glyceryl laurate (Gelucire 44/1®), PEG-32 glyceryl palmitostearate (Gelucire 50/13®), PEG-32 glyceryl stearate (Gelucire 53/10®), glyceryl behenate, cetyl palmitate, glyceryl di and tri stearate, glyceryl palmitostearate, and glyceryl triacetate (Triacetin®). The use of two or more plasticizers in a combination or blend of varying ratios is also encompassed by the present invention.

In order to prepare a BAS-loaded delivery system, the BAS can be loaded in any physical form (i.e. solid, liquid, gel or paste, where the BAS is dissolved or suspended in the plasticizer or mixtures of plasticizers, volatile solvents or mixture of volatile solvents or mixtures of volatile solvents and plasticizers) at any step during the manufacturing process of biodegradable delivery systems before the volatile solvent is completely removed. It can also be manufactured by loading the BAS soon after the biodegradable vehicle is prepared, or blending the BAS to the biodegradable vehicle just prior to the use of the BAS-loaded biodegradable delivery system. Mixing the BAS with the biodegradable vehicle can be accomplished by simply stirring the mixture with a stirring device, or by triturating the mixture or employing an ointment mill or a suitable device or apparatus or equipment that can be used for blending/mixing. When the BAS is blended with the biodegradable vehicle just prior to use, it could be stored in a separate container in a solid state, liquid state (where the BAS is dissolved or suspended in the plasticizer or blends of plasticizers), or gel or paste (where the BAS is dissolved or suspended in the plasticizer or blends of plasticizers). Alternatively, a device, which resembles two syringes or syringe-like devices (e.g. pumps in which materials can be mixed by depressing a trigger-like device) attached together with a removable partition or a valve assembly can also be used to uniformly mix the BAS with the biodegradable vehicle. The BAS is loaded in one syringe or compartment and the biodegradable vehicle is loaded in the other compartment. A removable partition or a valve, which will allow the contents of the two compartments to be mixed uniformly, separates the two compartments. The mixing process is performed in order to dissolve or uniformly suspend the BAS particles in the biodegradable vehicle. The resulting BAS-loaded biodegradable delivery systems can be free flowing or viscous liquids, gels or pastes. In order to prepare a BAS-loaded delivery system just prior to use, the BAS and the biodegradable vehicle can be packaged in two separate containers as a kit. The vehicle and the BAS can then be blended together by the aforementioned methods.

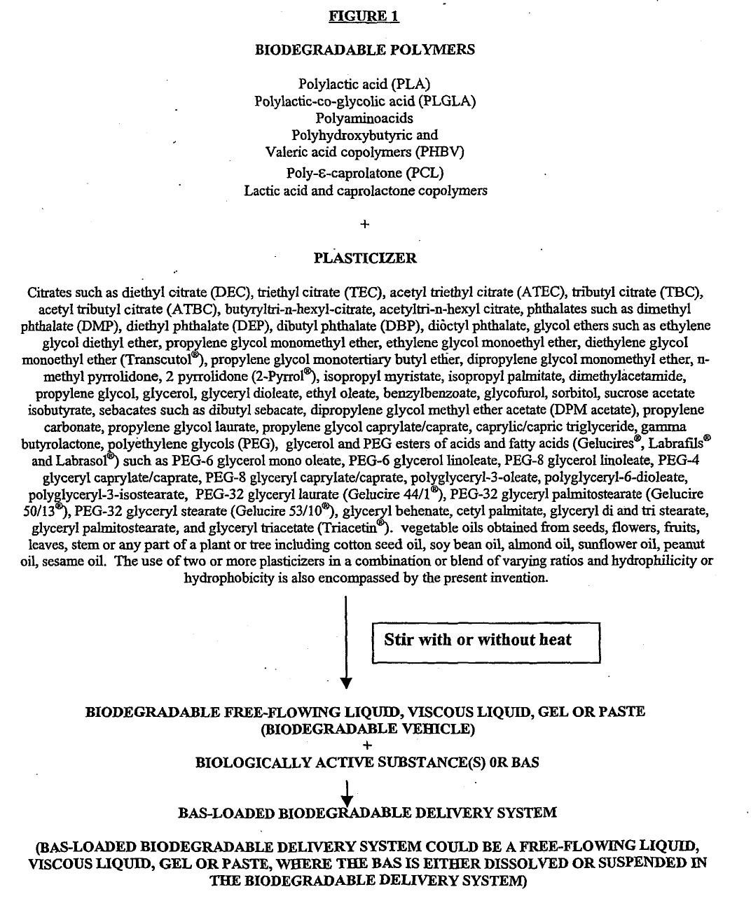

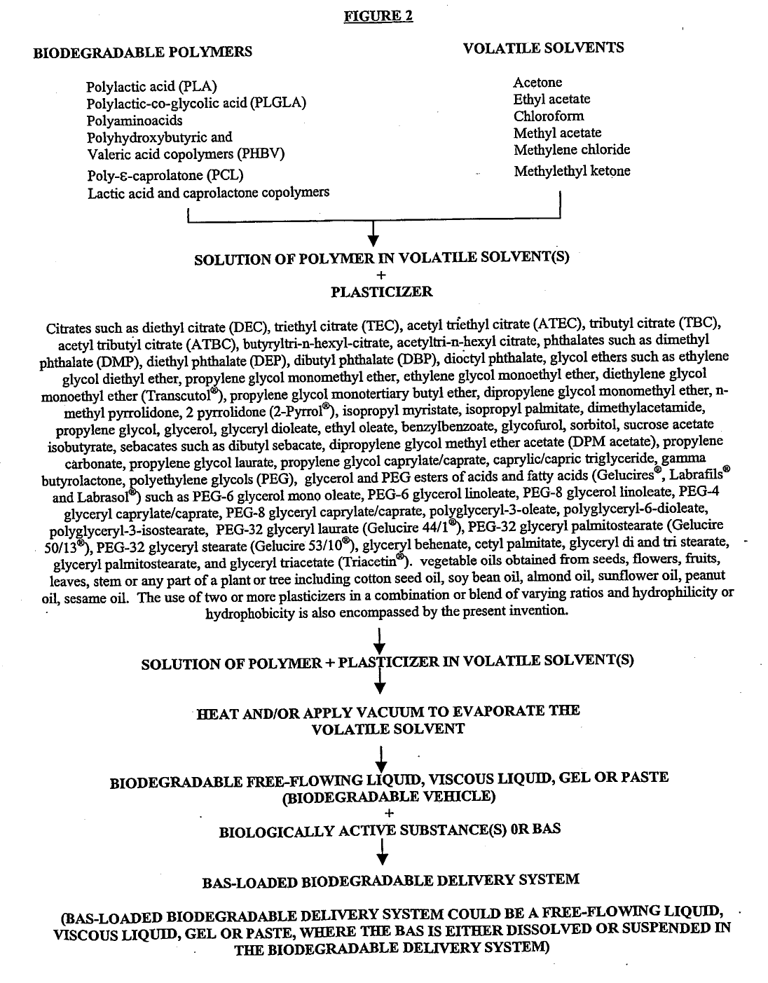

The procedure for preparing a biodegradable vehicle first, loading the BAS soon after the biodegradable vehicle is prepared, or blending the BAS to the biodegradable vehicle just prior to the use of the BAS-loaded biodegradable delivery system is shown in FIGS. 1 and 2.

The procedure of loading BAS before removing the volatile solvent or mixture of volatile solvents to prepare biodegradable delivery systems is shown in FIG. 2. However, the method of addition of the BAS is not limited to that shown in FIG. 2, since the BAS can be loaded in any physical form (i.e. solid, liquid, gel or paste, where the BAS is dissolved or suspended in the plasticizer or mixtures of plasticizers, volatile solvents or mixture of volatile solvents or mixtures of volatile solvents and plasticizers, at any step during the manufacturing process, before the volatile solvent is completely removed.

The resulting BAS-loaded biodegradable delivery systems can be free flowing or viscous liquids, gels or pastes, wherein the BAS can be dissolved or suspended.

Examples of BAS include, but are not limited to, steroids, hormones, antipsychotic agents, agents that act on the central nervous system (CNS-agents), narcotic agonists and antagonists, fertility regulating agents, antibodies and antigens, anesthetics, analgesics, antibiotics, antiviral agents, antineoplastic agents, antifungal agents, cavity and infection preventing agents, cardiovascular agents, angiogenic and antiangiogenic agents, anti-inflammatory agents, immunomodulators, vasodilators, brochiodilators, alkaloids, peptides and proteins, vaccines, live or killed-bacteria and viruses, agents or extracts derived from whole or parts of plants, trees, flowers, fruits, buds, seeds, leaves, barks, stem, roots, and animal tissues, growth promoting agents, soft and hard tissues, growth factors, human growth factor, human growth hormone, FGF, erythropoietin, Nupagen, granulocyte colony-stimulating factor (G-CSF), cells, tissues such as bones or agents derived there from, bone growth promoting agents such as calcium phosphates, calcium sulfate and hydroxyapatites, whole viable cells and cell-lines, genes, nucleic acid, antisense, deoxyribonucleic acid (DNA), DNA fragments, ribonucleic acid (RNA), RNA fragments, and biological tissues such as islets of langerhans and pancreas, insulin, vitamin and mineral supplements, iron, chelating agents, coagulants, anticoagulants, and the like.

In certain aspects, the bioactive agents include anticancer agents such as taxol, carmustine, interleukin 2, interferon, growth hormones such as human growth hormone, somatotropin hormone, antipsychotic agents such as risperidone, antibiotics such as gentamicin, tetracycline, oxytetracycline, topical anesthetic agents such as benzocaine, chloroprocaine, cocaine, procaine, propoxycaine tetracaine, depravaine, bupivacaine, etidocaine, levobupivacaine, lidocaine, mepivacaine, prilocaine, propofol and ropivacaine, analgesic agents such as morphine, oxycodone, fentanyl, fentanyl, sufentanyl, butorphanol, narcotic antagonists such as naltrexone, nalorphine, naloxone, nalmefene, growth promotic agents such as TGF alpha and TGF beta, bone morphogenic peptides and proteins and calcium salts such as calcium sulfate, calcium phosphate, and anti-inflammatory agents such as dichlofenac. In one preferred aspect, the present invention provides a biodegradable vehicle comprising oxytetracycline for veterinary use.

In certain other embodiments, the biologically active agents include, but are not limited to, steroids such as protaglandins, estrogens, androgens, and progestins; ophthalmics such as lubricants and anti-glaucoma; antibiotics such as quinolones; saliva subsitiutes, sedative/hypnotics such as benzodiazepines and barbituates; wound care such as growth factors (EPO, FGF, G-CSF); antiparasitics (worms, malarial); anticonvulsants, muscle relaxants, nucleoside analogs, osteoporosis preparations (supplement bone growth), antiparkinsonian agents, antibiotics such as cephalosporins, aminoglycosides and sulfonamides, oxytocic agents and prostaglandins.

Those of skill in the art will know of other biological agents useful in the practice of the present invention.

The physical form (i.e. liquids, gels or pastes), consistency or rheology, hydrophilicity or hydrophobicity, in vivo duration of stay of the biodegradable vehicles or delivery systems, in vivo biodegradation rate of biodegradable vehicles or delivery systems, and BAS release characteristics from BAS-loaded biodegradable delivery systems depend on a number of factors. These include: type of polymer or copolymer, hydrophilicity or lipophilicity of polymer or copolymer, concentration of polymer or copolymer, molecular weight of polymer or copolymer, copolymer ratios, combination of polymers or copolymers with different molecular weights, combination of copolymer with varying copolymer ratios, combination of different types of polymer with varying crystallinity, hydrophilicity or hydrophobicity, type of plasticizer, hydrophilicity or lipophilicity of plasticizer, concentration of plasticizer (polymer or copolymer to plasticizer/plasticizers ratios), combination of plasticizers, type of BAS, loading of BAS, hydrophilicity or lipophilicity of BAS, molecular weight of BAS. In addition, the physicochemical interactions between the polymer, plasticizer and BAS also affect the above-mentioned properties of biodegradable vehicles and delivery systems.

For example, using the present invention, it is possible to tailor the release of a BAS (with specific physicochemical properties and the desired in vivo concentration), for the desired length time. This is achieved by blending an appropriately selected polymer or polymers with an appropriately selected plasticizer or mixtures of plasticizers. Besides controlling the release characteristics of the BAS from the delivery system described in the present invention, a blend of the appropriate polymer or polymers and plasticizer also controls the consistency or rheology of the delivery system.

It is also possible to extend the in vivo duration of stay of the biodegradable vehicle or delivery system by selecting a higher molecular weight or highly hydrophobic polymer, since polymers with higher molecular weights or high hrdrophobicity generally degrade slowly in the body. Furthermore, it is possible to modify the degradation kinetics of the biodegradable vehicle or delivery system, or obtain pulsatile or intermittently fluctuating delivery of the BAS from the BAS-loaded delivery systems by combining polymers of different molecular weights (e.g. low, intermediate and high molecular weights or low and high molecular weights or low and medium molecular weights or medium and high molecular weights), whereby the low molecular weight polymer in the biodegradable vehicle may degrade at a much faster rate than the rest of the polymer in the blend. Alternatively, using blends of copolymers of different copolymer ratios of varying hydrophilicity and hydrophobicity (e.g. different copolymer ratio of lactide-glycolide or lactide-caprolactone) or using blends of two different polymers or copolymers with different crystallinity (e.g. blends of polyacaprolactone and polylactic acid or polycaprolactone and poly-lactic-co-glycolic acid/polylactide-co-glycolide (PLGA)) can also result in a biodegradable vehicle or biodegradable delivery system with varying degradation kinetics where the more hydrophilic or amorphous polymer may degrade at a much faster rate than the rest of the polymers in the blend.

The biodegradable vehicle without any BAS may be used as a biodegradable tissue or cavity filler or spacer in the body, whereas, BAS-loaded biodegradable delivery system may be used for the treatment of a variety of diseases and pathological conditions. The final composition with or without the BAS may be injected, implanted, smeared or applied in animals, birds or humans.

For example, the biodegradable delivery system loaded with an antitumor agent or antiangiogenic agent can be directly injected into or adjacent to solid tumors such as brain tumor, breast tumors, melanomas, etc. It can also be injected, implanted or smeared at a site from where a solid tumor has been surgically removed, thus affording site-specific delivery for disease states that are otherwise very difficult, (if not impossible) to treat using the conventional methods of treatment. For localized BAS delivery and treatment, BAS-loaded biodegradable vehicle can also be used in surgeries where appropriate quantities of an antibiotic, an anti-inflammatory agent, a local anesthetic or analgesic, or combinations thereof can be loaded in the biodegradable vehicle by the surgeon in an operating room, and the resulting mixture can then be injected, implanted, smeared or applied at the site of surgery to minimize the chances of localized infections or inflammation and reduce pain respectively, due to surgery. In the case of orthopedic surgery, currently, the majority of the orthopedic surgeons prepare beads in the operating room with a non-biodegradable polymer, polymethylmethacrylate (PMMA). These beads are loaded with an appropriate dose of an antibiotic. These beads are then placed in the cavity at the site of surgery to prevent infections such as osteomyelitis. However, the non-degradable polymer beads have to be eventually removed before closing the wound with a suture, and the patients are then given an intravenous dose of an antibiotic or treated with an oral antibiotic. This procedure can easily be corrected with the use of an antibiotic loaded biodegradable vehicle that can be injected, implanted, smeared or applied near or at the site of surgery. High concentrations of the antibiotic at the site of surgery can prevent infections. Moreover, the BAS delivery system need not be removed from the site of administration because of the biodegradable nature of the system. The biodegradable vehicle loaded with bone growth promoting agents such as calcium sulfate, calcium phosphate or hydroxyapatite can be injected, implanted, applied or smeared at an appropriate site, where it is needed following bone, disc or spine surgery. BAS such as low molecular weight heparin can also be incorporated into the biodegradable vehicle and the resulting mixture can be used to treat conditions such as deep venous thrombosis (DVT) in trauma or surgical patients.

The system could be loaded with a contraceptive agent, antipsychotic agent, anticonvulsants, antimalarial, antihypertensive agent, antibiotics, antiviral agents, biologically active protein and peptides, vaccines, live or killed bacteria and viruses, genes, DNA or DNA fragments, RNA or RNA fragments, and injected, implanted, smeared or applied in the body to provide a controlled release of the agents for the desired length of time. Biodegradable delivery system loaded with BAS such as antiinflammatory agents, analgesics and anesthetics could be injected directly into joints or sites in the body from where the pain is emanating, thus providing relief from the excruciating pain and making the joints more mobile. Antigens may also be incorporated into the delivery system and injected, implanted or applied in animals or humans to induce the production of specific antibodies. Bones (fragments or powder), morphogenic proteins such as growth promoting agents of biological tissues and organs and wound-healing factors, can also be incorporated into the biodegradable vehicle, and the resulting mixture is injected, implanted or applied at the site of administration. Live cells and/or whole or a part of a tissue or tissues and organs can also be blended with the biodegradable vehicle and injected, implanted or applied at the site of administration. For pulsatile or intermittent delivery of BAS such as vaccines, the biodegradable vehicle can be prepared with blends of varying molecular weights of polymers or copolymers, or with blends of copolymers of varying copolymer ratios (e.g. 50/50 PLGA and 85/15 PLGA or 100% PLA and 25/75 PLGA) or blends of different types of biodegradable polymers with varying hydrophobicity or lipophilicity or crystallinity (e.g. 1:1 of PLA:PCL or 1:3 of PLA:PCL or 1:1 of 50/50 PLGA:PCL).

The formulation, which is sterile, is suitable for various topical or parenteral routes, such as intramuscular, subcutaneous, intra-articular, by suppository (e.g. per-rectum or vaginal application), intradermal. In certain aspects, the biological active agents and biodegradable delivery systems are delivered or administered topically. Additionally, the agents can be delivered parenterally. Topical administration is preferred in treatment of lesions of the skin as in psoriasis, where such direct application is practical and clinically indicated.

An effective quantity of the compound of interest is employed in treatment. The dosage of compounds used in accordance with the invention varies depending on the compound and the condition being treated. For example, the age, weight, and clinical condition of the recipient patient; and the experience and judgment of the clinician or practitioner administering the therapy are among the factors affecting the selected dosage. Other factors include: the route of administration, the patient, the patient's medical history, the severity of the disease process, and the potency of the particular compound. The dose should be sufficient to ameliorate symptoms or signs of the disease treated without producing unacceptable toxicity to the patient. In general, an effective amount of the compound is that which provides either subjective relief of symptoms or an objectively identifiable improvement as noted by the clinician or other qualified observer.

This invention will be understood with greater particularity by reviewing the following examples:

### EXAMPLES

### Example 1

Preparation of a Biodegradable Vehicle:

A polymer (50% w/w of 50/50 lactide-co-glycolide copolymer) was dissolved in minimum quantity of acetone. Triethyl citrate (TEC), at a concentration of 50% w/w, was added to the polymer solution and was stirred to yield a uniform mixture. Acetone was evaporated from the mixture by heating at 60-75° C. with constant stirring. The resulting formulation obtained was a matrix with a gel-like consistency.

### Example 2

Example 1 was repeated using 10% w/w of 50/50 lactide-co-glycolide copolymer and 90% w/w TEC. The resulting formulation obtained was a matrix with a liquid-like consistency.

### Example 3

Example 1 was repeated using 20% w/w of 50/50 lactide-co-glycolide copolymer and 80% w/w TEC. The resulting formulation obtained was a matrix with a viscous liquid-like consistency.

### Example 4

Example 1 was repeated, using 30% w/w of 50/50 lactide-co-glycolide copolymer and 70% w/w TEC was used. The resulting formulation obtained was a matrix with a viscous liquid-like consistency.

### Example 5

Example 1 was repeated, using 40% w/w of 50/50 lactide-co-glycolide copolymer and 60% w/w TEC was used. The resulting formulation obtained was a matrix with a viscous liquid-like consistency.

### Example 6

Example 1 was repeated, using 60% w/w of 50/50 lactide-co-glycolide copolymer and 40% w/w TEC was used. The resulting formulation obtained was a matrix with a gel-like consistency.

### Example 7

Example 1 was repeated, using 70% w/w of 50/50 lactide-co-glycolide copolymer and 30% w/w TEC was used. The resulting formulation obtained was a matrix with a gel-like consistency.

### Example 8

Example 1 was repeated, using 80% w/w of 50/50 lactide-co-glycolide copolymer and 20% w/w TEC was used. The resulting formulation obtained was a matrix with thick sticky paste.

### Example 9

Example 1 was repeated with the following polymers and plasticizers as shown in Table 1 below:

TABLE 1

| | | | DESCRIPTION OF THE |
|---|---|---|---|
| TYPE OF POLYMER | PLASTICIZER | SOLVENT | FORMULATION |
| DL-POLYLACTIC ACID | GLYCERYL TRIACETATE | ACETONE | GEL, SLIGHTLY CLOUDY |
| (DL-PLA; I.V. = 0.58) | (TRIACETIN) | | |
| DL-POLYLACTIC ACID | TRIETHYL CITRATE | ACETONE | GEL, TRANSPARENT |
| (DL-PLA; I.V. = 0.58) | (TEC) | | |
| DL-POLYLACTIC ACID | ACETYL TRIETHYL CITRATE | ACETONE | GEL, SLIGHTLY CLOUDY |
| (DL-PLA; I.V. = 0.58) | (ATEC) | | |
| DL-POLYLACTIC ACID | DIMETHYL PHTHALATE | ACETONE | GEL, LESS VISCOUS, |
| (DL-PLA; I.V. = 0.58) | (DMP) | | TRANSPARENT |
| DL-POLYLACTIC ACID | DIETHYL PHTHALATE | ACETONE | GEL, TRANSPARENT |
| (DL-PLA; I.V. = 0.58) | (DEP) | | |
| DL-POLYLACTIC-CO-GLYCOLIC ACID | GLYCERYL TRIACETATE | ACETONE | GEL, LESS VISCOUS, |
| (DL-PLGA; I.V. = 0.58) | (TRIACETIN) | | SLIGHTLY YELLOW |
| DL-POLYLACTIC-CO-GLYCOLIC ACID | TRIETHYL CITRATE | ACETONE | GEL, SLIGHTLY YELLOW |
| (DL-PLGA; I.V. = 0.58) | (TEC) | | |
| DL-POLYLACTIC-CO-GLYCOLIC ACID | ACETYL TRIETHYL CITRATE | ACETONE | GEL, SLIGHTLY YELLOW |
| (DL-PLGA; I.V. = 0.58) | (ATEC) | | |
| DL-POLYLACTIC-CO-GLYCOLIC ACID | TRIETHYL CITRATE | ACETONE | GEL, SLIGHTLY YELLOW |
| (DL-PLGA; I.V. = 0.58) | (TEC) | | |
| DL-POLYLACTIC-CO-GLYCOLIC ACID | DIMETHYL PHTHALATE | ACETONE | GEL, LESS VISCOUS, |
| (DL-PLGA; I.V. = 0.58) | (DMP) | | RANSPARENT |
| DL-POLYLACTIC-CO-GLYCOLIC ACID | DIETHYL PHTHALATE | ACETONE | GEL, SLIGHTLY YELLOW |
| (DL-PLGA; I.V. = 0.58) | (DEP) | | |
| DL-POLYLACTIC-CO-GLYCOLIC ACID | N-METHYL PYRROLIDONE | ACETONE | VISCOUS LIQUID, |
| (DL-PLGA; I.V. = 0.58) | (NMP) | | TRANSPARENT |
| DL-POLYLACTIC-CO-GLYCOLIC ACID | GLYCERYL TRIACETATE | ACETONE | VISCOUS LIQUID, |
| (DL-PLGA I.V. = 0.15) | (TRIACETEN) | | TRANSPARENT |
| DL-POLYLACTIC-CO-GLYCOLIC ACID | TRIETHYL CITRATE | ACETONE | VISCOUS LIQUID, |
| (DL-PLGA; I.V. 0.15) | (TEC) | | TRANSPARENT |
| DL-POLYLACTIC-CO-GLYCOLIC ACID | ACETYL TRIETHYL CITRATE | ACETONE | VISCOUS LIQUID, |
| (DL-PLGA; I.V. 0.15) | (ATEC) | | TRANSPARENT |
| DL-POLYLACTIC-CO-GLYCOLIC ACID | TRIETHYL CITRATE | ACETONE | VISCOUS LIQUID, |
| (DL-PLGA; I.V. = 0.15) | (TEC) | | TRANSPARENT |

### Example 10

Several polymers were separately dissolved in several volatile solvents. Several plasticizers were separately added to the polymer-solutions, such that the ratio of polymer to plasticizer in the final formulations ranged from 1:19 to 4:1. Several drugs were separately added to the polymer-plasticizer-solvent blends. The solvents were then evaporated at an elevated temperature to obtain drug-loaded formulations. The drug content in the final formulations constituted up to 50% w/w.

For several formulations, blank formulations of polymers and plasticizers blends were first obtained. The drugs were then separately added to the blank formulations to obtain drug-loaded formulations. Table 2 lists examples of polymers, plasticizers, solvents, polymer to plasticizer ratio and concentration of drugs in the formulations.

TABLE 2

| | | | | | CONCEN- |
|---|---|---|---|---|---|
| | | | | | TRATION |
| | | | | | OF DRUGS |
| | | | POLYMER | | (% w/w) |
| | | | TO | | IN |
| TYPE OF | | | PLASTICIZER | | POLYMER |
| POLYMERS | PLASTICIZERS | SOLVENTS | RATIOS | DRUGS | MATRICES |
| POLYCAPROLAC- | DIETHYLENE | METHYLENE | 1:1 | TESTOSTERONE | 0.5%-50% |
| TONE | GLYCOL | CHLORIDE | | | w/w |
| POLYLACTIC ACID | MONOETHYL ETHER | CHLOROFORM | 1:2 | PROGESTERONE | |
| | (TRANSCUTOL ®), | | 1:3 | LEVONORGESTREL | |
| POLYLACTIC-CO- | PEG-8-GLYCERYL | ACETONE | 1:4 | THEOPHYLLINE | |
| GLYCOLIC ACID | CAPRYLATE/CAPRA | | | | |
| | TE | ETHYL ACETATE | | | |
| COPOLYMERS OF | (LABRASOL ®) | | 1:9 | PROPRANOLOL | |
| LACTIC ACID AND | TRIETHYL CITRATE | | 1:19 | ATENOLOL | |
| CAPROLACTONE | (TEC), | | | | |
| | ACETYL TRIETHYL | | 2:1 | METOPROLOL | |
| | CITRATE (ATEC) | | 2:3 | CHLORPROAMAZINE | |
| | GLYCERYL | | 3:2 | CLONIDINE | |
| | TRIACETATE | INSULIN | | | |
| | (TRIACETIN ®) | | 3:1 | | |
| | POLYETHYLENE | | 4:1 | OXYTETRACYCLINE | |
| | GLYCOLS (PEG) | | | NALTREXONE | |
| | N-METHYL | | | | |
| | PYRROLIDONE | | | | |
| | (NMP) | | | | |

### Example 11

Effect of Varying Polymer-to-Plasticizer Ratios on the Physical State of Formulations and Drug Release Characteristics

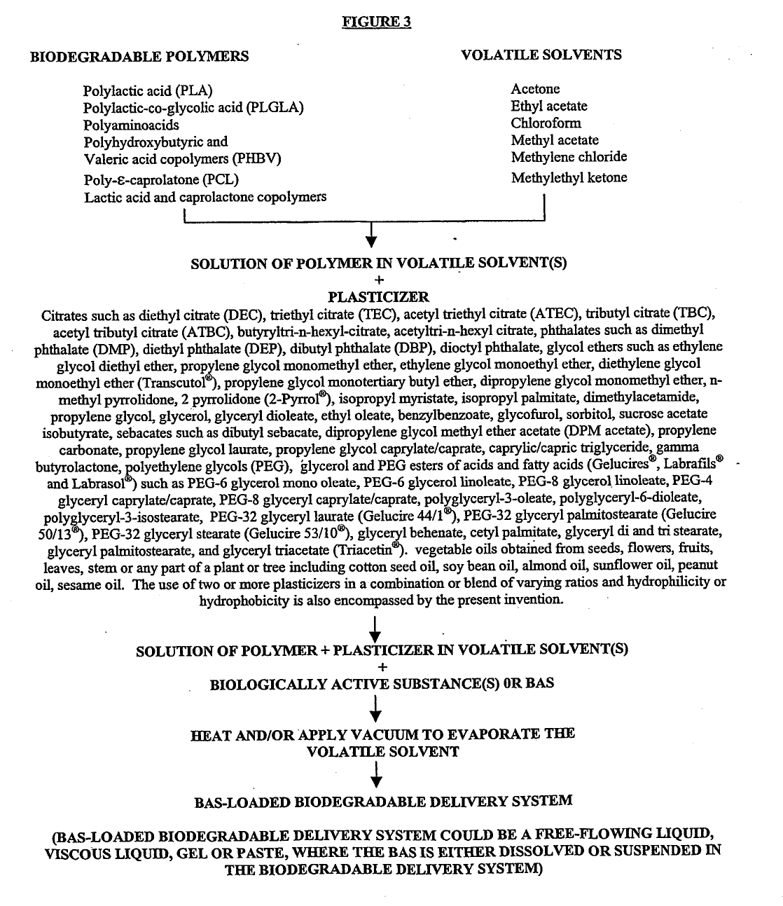

Several samples of polylactic-co-glycolic acid (inherent viscosoty—0.59) were weighed and separately dissolved in acetone. Varying ratios of N-methyl pyrrolidone (NMP) were separately added to the polymer-solutions, such that the ratio of polymer to plasticizer in the formulations ranged from 20:80 to 80:20. Acetone was then evaporated by heating the solutions at 70-80° C. Levonorgestrel (2% w/w) was added to the resulting formulations. Table 3 describes the physical state of the formulations containing varying polymer-to-plasticizer ratios. Drug release characteristics from the formulations depicted in Table 3 are shown in FIG. 3.

TABLE 3
Physical state of formulations prepared with varying polymer-to-plasticizer
ratios

| | Physical State of the | Physical State of Drug in |
|---|---|---|
| Polymer*-to-NMP Ratio | Formulation | the Formulation |
| 20:80 | Very flowable liquid | Dissolved |
| 40:60 | Viscous liquid | Dissolved initially; |
| | | however precipitated |
| | | partially after 48 hrs |
| 50:50 | Flowable gel | Suspended |
| 60:40 | Flowable gel | Suspended |
| 80:20 | Thick paste | Suspended |

*50/50 Polylactide-co-glycolide (IV = 0.59 dL/g)
Drug loading = 2% w/w

### Example 12

Effect of Varying Polymer Inherent Viscosities on the Physical State of the Formulations and Drug Release Characteristics

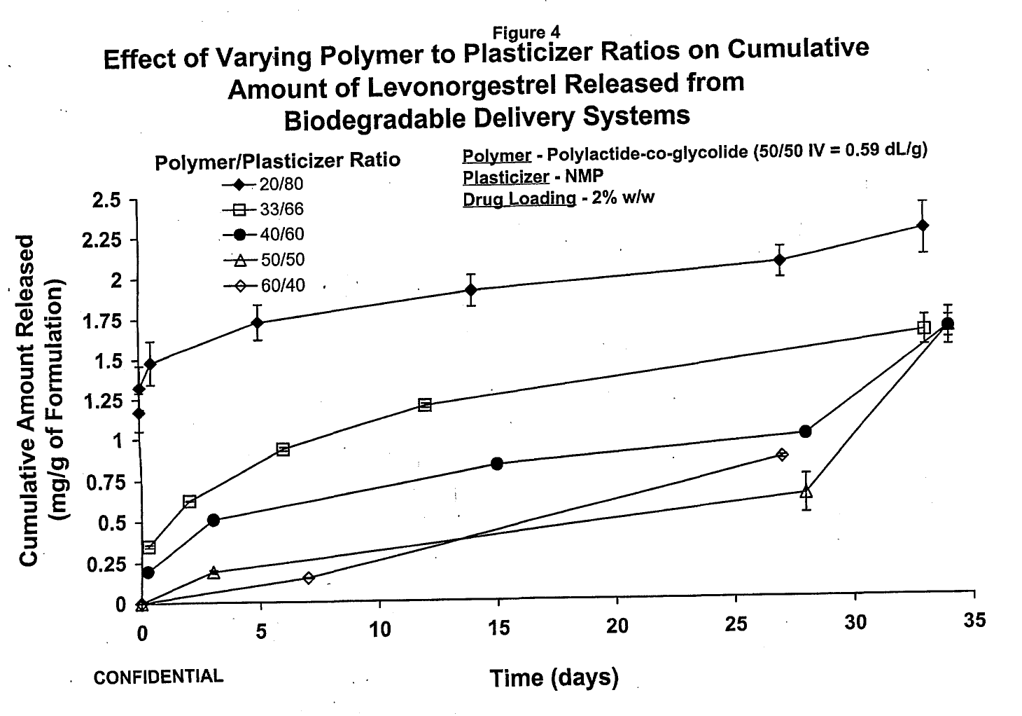

Several samples of polylactic-co-glycolic acid (PLGA) with varying inherent viscosities ranging from 0.15-1.07) were weighed and separately dissolved in acetone. An appropriate quantity of N-methyl pyrrolidone (NMP) was added to the polymer-solutions such that the ratio of polymer to plasticizer in the formulations was 33% PLGA and 67% NMP. Acetone was then evaporated by heating the solutions at 70-80° C. Levonorgestrel (2% w/w) was added to the resulting formulations. Table 4 describes the physical state of the formulations containing varying polymer inherent viscosities. Drug release characteristics from the formulations depicted in Table 4 are shown in FIG. 4.

TABLE 4
Physical state of formulations prepared with polymer of varying inherent
viscosities

| Polymer Inherent | Physical State of the | Physical State of Drug in |
|---|---|---|
| Viscosity (dL/g) | Formulation* | the Formulation* |
| 0.15 | Very flowable liquid | Dissolved |
| 0.26 | Flowable liquid | Dissolved |
| 0.42 | Flowable liquid | Dissolved |
| 0.59 | Viscous liquid | Dissolved |
| 0.74 | Flowable gel | Dissolved |
| 1.07 | Viscous gel | Dissolved |

*33% w/w of 50/50 Polylactide-co-glycolide and 67% w/w NMP
Drug loading = 2% w/w

### Example 13

Effect of Varying Copolymer Ratios on Physical State of Formulations and Drug Release Characteristics

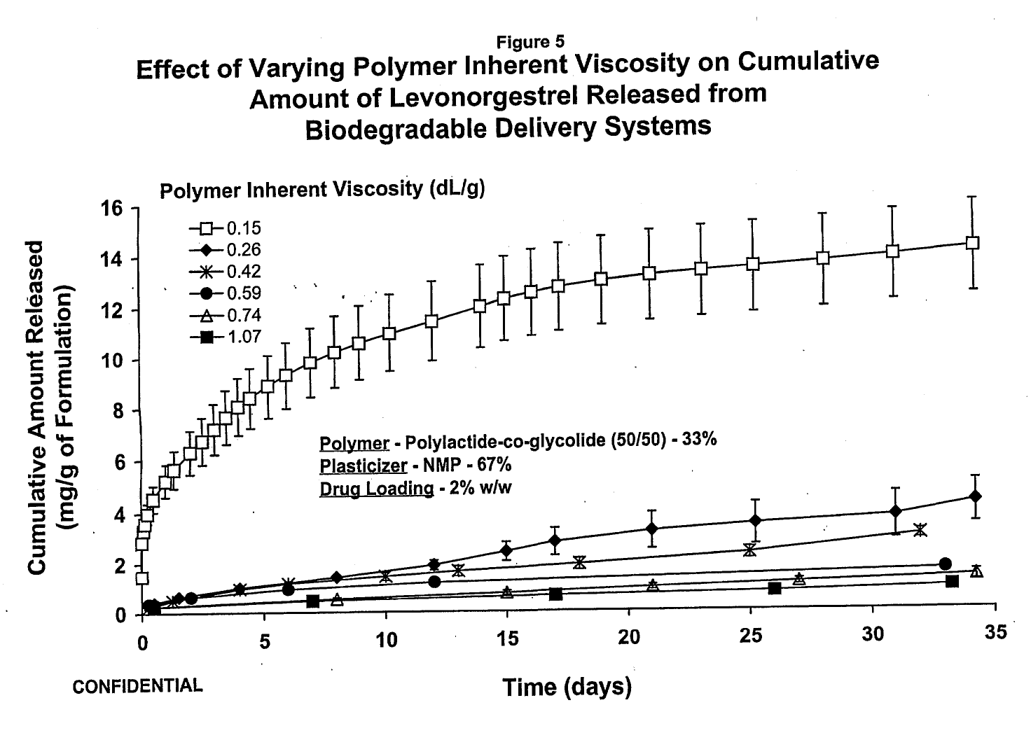

Samples of pure polylactic acid and polylactic-co-glycolic acid (PLGA) with varying copolymer ratios ranging from 50/50 to 85/15 were weighed and separately dissolved in acetone. An appropriate quantity of N-methyl pyrrolidone (NMP) was added to the polymer-solutions such that the ratio of polymer to plasticizer in the formulations was 33% PLGA and 67% NMP. Acetone was then evaporated by heating the solutions at 70-80° C. Levonorgestrel (2% w/w) was added to the resulting formulations. Table 5 describes the physical state of the formulations prepared from varying copolymer ratios. Drug release characteristics from the formulations depicted in Table 5 are shown in FIG. 5.

TABLE 5
Physical state of formulations prepared with polymers of varying
copolymer ratios.

| Ratio of Lactide to | | |
|---|---|---|
| Glycolide in | Physical State of the | Physical State of Drug in |
| Polymer | Formulation* | the Formulation* |
| 50/50 | Yellowish, viscous | Dissolved |
| | liquid | |
| 65/35 | Yellowish, viscous | Dissolved |
| | liquid | |
| 75/25 | Pale yellow, highly | Dissolved |
| | viscous liquid | |
| 85/15 | Straw colored, slightly | Dissolved |
| | translucent, highly | |
| | viscous liquid | |
| 100/0 | Clear, highly viscous | Dissolved |
| | liquid | |

*33% w/w of Polylactide-co-glycolide and 67% w/w NMP
Drug loading = 2% w/w

### Example 14

Effect of Varying Drug Loadings on Drug Release

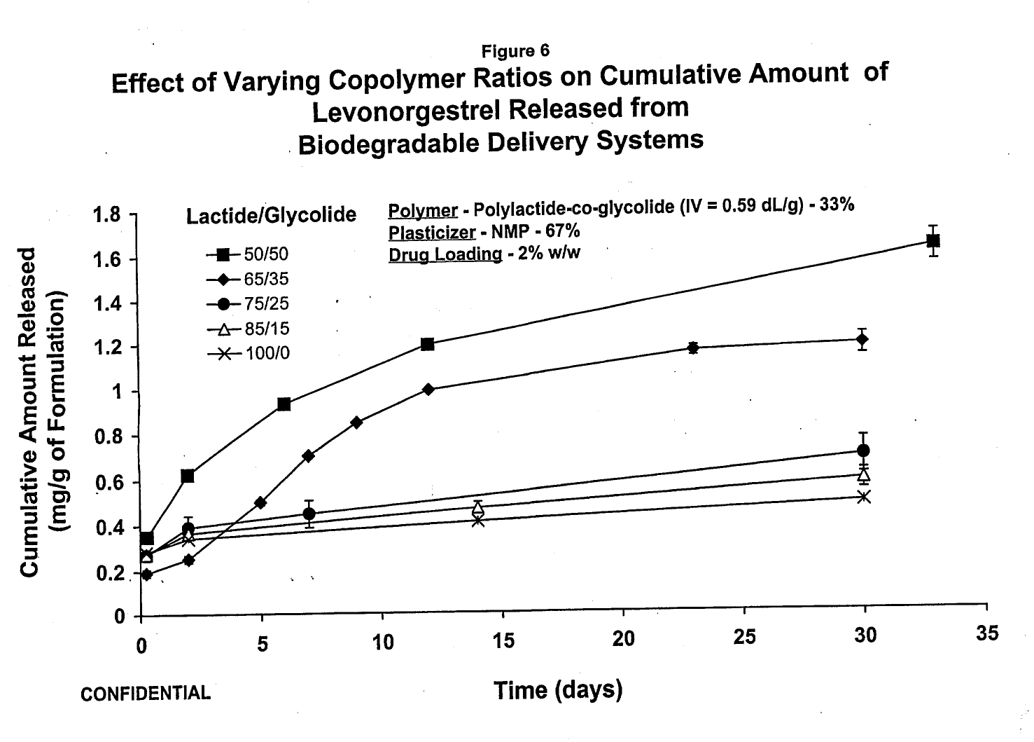

A polymer (25% w/w of 50/50 lactide-co-glycolide copolymer, inherent viscosity of 0.59) was dissolved in a minimum quantity of acetone. Pure polyethylene glycol 400 (PEG 400) was added to the polymer solution. The solution was stirred to yield a uniform mixture. Acetone was evaporated from the mixture by heating at 60-75° C. with constant stirring. The blank formulation was kept in a vacuum oven at 60-75° C. overnight to ensure complete removal of acetone. The resulting formulation obtained was a matrix with a viscous liquid like consistency. Three different concentrations of oxytetracycline base (either 10, 20 or 30% w/w) were added to the blank formulation and mixed thoroughly to ensure uniform distribution of the drug in the formulations. Drug release from the drug-loaded formulations was performed at 37° C. in isotonic phosphate buffer containing sodium sulfite as an antioxidant. FIG. 6 shows the cumulative amount of oxytetracycline released from formulations prepared with the above-mentioned compositions. Increasing the percentage of drug in the formulations from 10 to 30% w/w increased the cumulative amount of drug released at the end of 360 hours. This increase occurred because, at higher drug-loadings, more drug is available on the surface of the formulations for release. Moreover, a higher drug concentration gradient between the formulation and the dissolution medium is expected at 30% w/w drug-loading compared to the one at 10% w/w drug loading.

### Example 15

Effect of Plasticizer Compositions on Drug Release

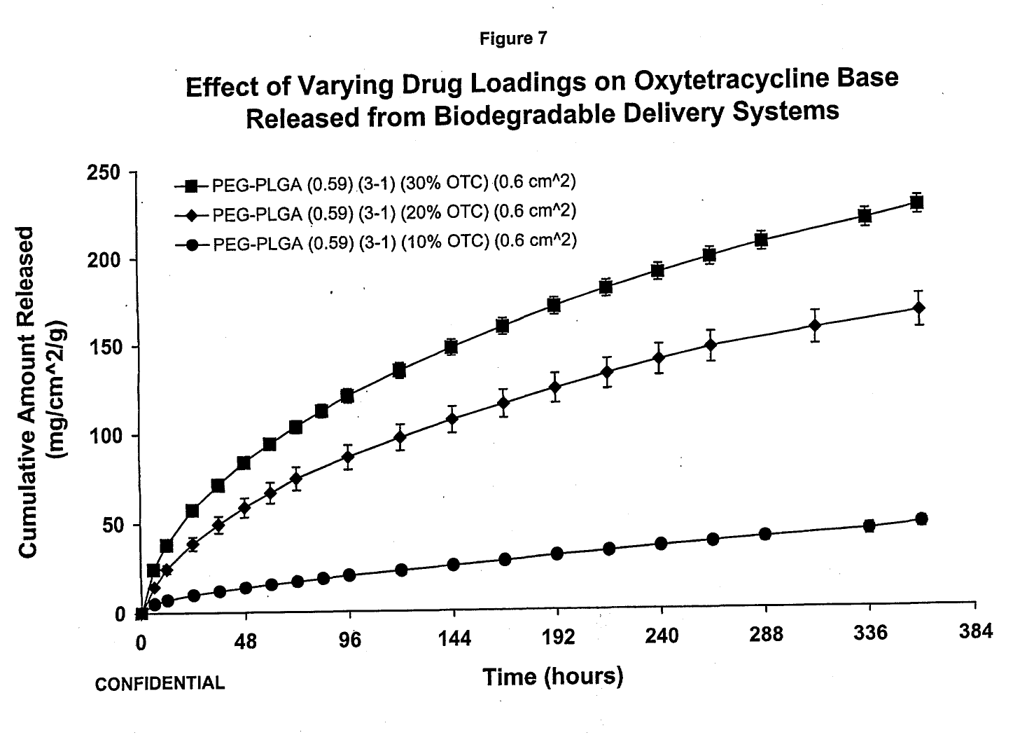

A polymer (25% w/w of 50/50 lactide-co-glycolide copolymer, inherent viscosity of 0.59) was dissolved in a minimum quantity of acetone. Either pure triethyl citrate (TEC), or polyethylene glycol 400 (PEG 400), or blends of PEG 400 and TEC (either 50/50% or 75/25% blends of PEG 400/TEC) was added to the polymer solution. The resulting solutions were stirred to yield uniform mixtures. Acetone was evaporated from the mixtures by heating at 60-75° C. with constant stirring. The blank formulations were kept in a vacuum oven at 60-75° C. overnight to ensure complete removal of acetone. The resulting formulations obtained were matrices with a viscous liquid like consistency. Oxytetracycline base (20% w/w) was added to each blank formulation and mixed thoroughly to ensure uniform distribution of the drug in the formulations. Drug release from the drug-loaded formulations was performed at 37° C. in isotonic phosphate buffer containing sodium sulfite as an antioxidant. FIG. 7 shows the cumulative amount of oxytetracycline released from formulations prepared with the above-mentioned compositions. Increasing the percentage of PEG 400 in the formulations prepared from 0% PEG 400 and 100% TEC to 100% PEG 400 and 0% TEC resulted in faster drug release. This is because PEG 400 is very hydrophilic and is completely miscible in water, whereas, the aqueous solubility of TEC is approximately 6%.

### Example 16

Effect of Varying Ratios of Polymer and Plasticizer on Drug Release

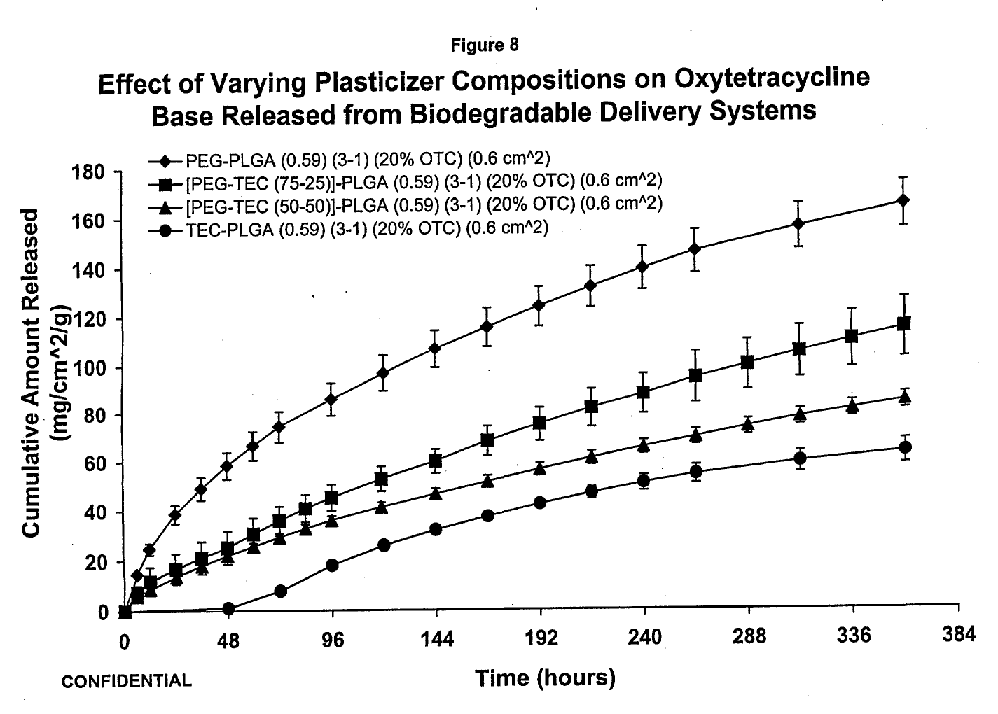

Three different concentrations (10, 20 or 25% w/w) of a polymer (50/50 lactide-co-glycolide copolymer, inherent viscosity of 0.59) were dissolved in a minimum quantity of acetone. Pure PEG 400 (90, 80 or 75% w/w) was added to the polymer solutions. The solutions were stirred to yield uniform mixtures. Acetone was evaporated from the mixtures by heating at 60-75° C. with constant stirring. The blank formulations were kept in a vacuum oven at 60-75° C. overnight to ensure complete removal of acetone. The resulting formulations obtained were matrices with varying viscosities or consistency. The formulation with 25% polymer was considerably more viscous than the one with 10% polymer. Oxytetracycline base (20% w/w) was added to each blank formulation and mixed thoroughly to ensure uniform distribution of the drug in the formulations. Drug release from the drug-loaded formulations was performed at 37° C. in isotonic phosphate buffer containing sodium sulfite as an antioxidant. FIG. 8 shows the cumulative amount of oxytetracycline released from formulations prepared with the above-mentioned compositions. It is evident from the figure that decreasing the percentage of polymer in the formulations from 25% to 10% dramatically increased the drug release. This is because a decrease in polymer concentration from 25% to 10% and a corresponding increase in the plasticizer concentration from 75% to 90% resulted in a decrease in the glass transition temperature, viscosity and an increase in polymer chain mobility of the formulations. Hence, the formulation with 10% polymer offered considerably less resistance for drug diffusion through the matrix compared to the one prepared with 25% polymer.

### Example 17

Effect of Varying Plasticizer Hydrophilicity on Drug Release

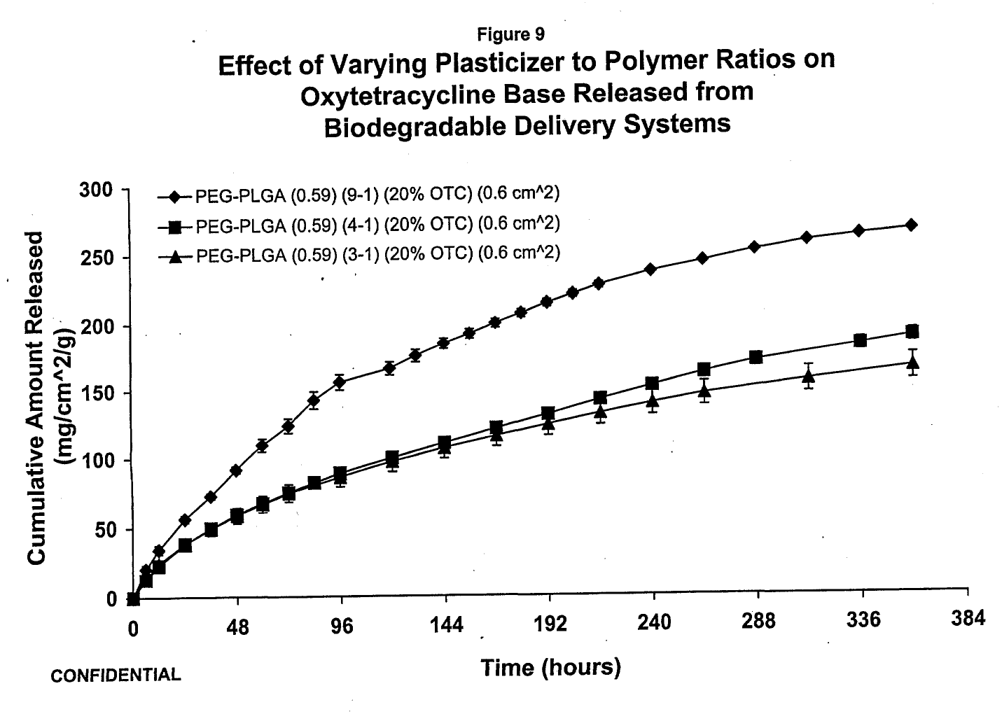

A polymer (25% w/w of 50/50 lactide-co-glycolide copolymer, inherent viscosity of 0.59) was dissolved in a minimum quantity of acetone. Either pure polyethylene glycol 400, triethyl citrate (TEC) or acetyl triethyl citrate (ATEC) was added to the polymer solution. The resulting solutions were stirred to yield uniform mixtures. Acetone was evaporated from the mixtures by heating at 60-75° C. with constant stirring. The blank formulations were kept in a vacuum oven at 60-75° C. overmight to ensure complete removal of acetone. The resulting formulations obtained were matrices with a viscous liquid like consistency. Oxytetracycline base (20% w/w) was added to each blank formulation and mixed thoroughly to ensure uniform distribution of the drug in the formulations. Drug release from the drug-loaded formulations was performed at 37° C. in isotonic phosphate buffer containing sodium sulfite as an antioxidant. FIG. 9 shows the cumulative amount of oxytetracycline released from formulations prepared with the above-mentioned compositions. It is evident from the figure that drug release was fastest from formulations prepared with PEG 400, and slowest from those prepared with ATEC. Intermediate drug release was observed from formulations prepared from TEC. This is because PEG 400 is completely miscible with water, whereas, the solubility of TEC in water is approximately 6% and ATEC is almost insoluble in water with an aqueous solubility of less than 0.1%.

### Example 18

Effect of Varying Polymer to Plasticizer Ratios and Plasticizer Compositions on Drug Release

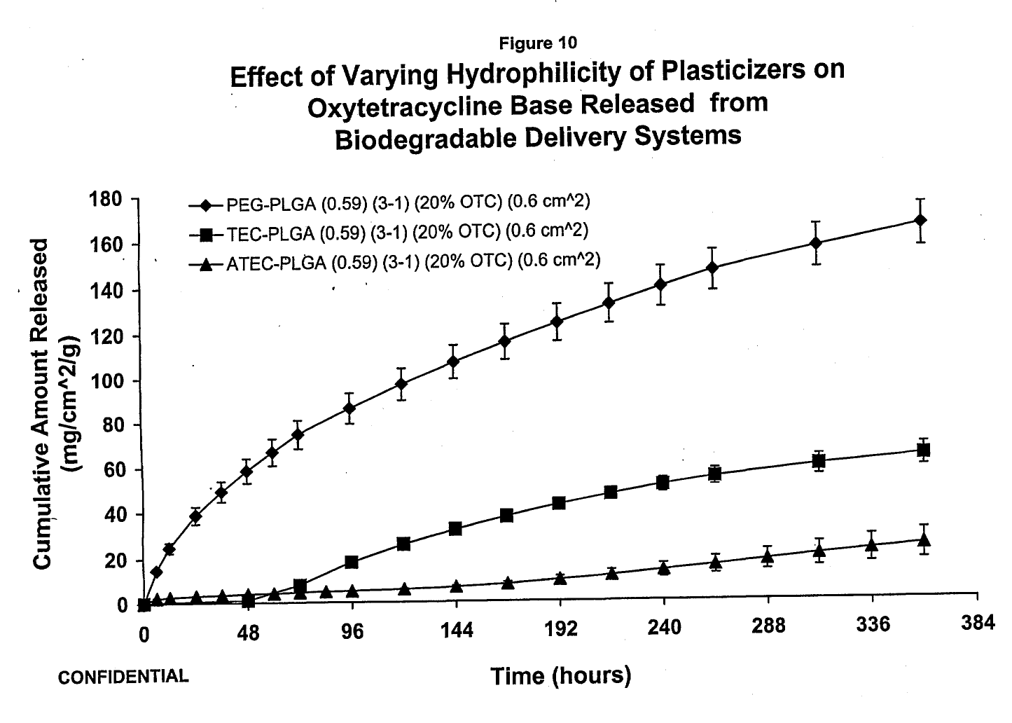

Blank formulations were prepared by dissolving either 16.67% w/w or 25% w/w of 50/50 polylactide-co-glycolide copolymer (inherent viscosity of 0.59) and either 50/50% or 75/25% blends of PEG 400 and TEC in a minimum quantity of acetone. The resulting solutions were stirred to yield uniform mixtures. Acetone was evaporated from the mixtures by heating at 60-75° C. with constant stirring. The blank formulations were kept in a vacuum oven at 60-75° C. overnight to ensure complete removal of acetone. The resulting formulations obtained were matrices with a viscous liquid like consistency. Oxytetracycline base (20% w/w) was added to each blank formulation and mixed thoroughly to ensure uniform distribution of the drug in the formulations. Drug release from the drug-loaded formulations was performed at 37° C. in isotonic phosphate buffer containing sodium sulfite as an antioxidant. FIG. 10 shows the cumulative amount of oxytetracycline released from formulations prepared with the above-mentioned compositions. It is evident from the figure that faster drug release was observed from formulations prepared with a 16.67% polymer and 83.3% of plasticizer blends of varying compositions (polymer to plasticizer ratio of 1:5) compared to those prepared from formulations with polymer to plasticizer ratios of 1:3 (25% polymer and 75% plasticizer). This is because increasing the polymer concentration in the formulations from 16.67% to 25% increased the viscosity of the formulations and decreased the drug diffusion from the formulations. Moreover, a comparison of drug released from formulations with similar polymer to plasticizer ratios but varying plasticizer compositions revealed that drug release was considerably faster from formulations prepared with blends of 75% PEG 400 and 25% TEC compared to those prepared from 50/50% blend of PEG 400/TEC. This is because the PEG 400 is completely miscible in water, whereas, the aqueous solubility of TEC in water is approximately 6%.

### Example 19

Effect of Varying Polymer Inherent Viscosities on Drug Release

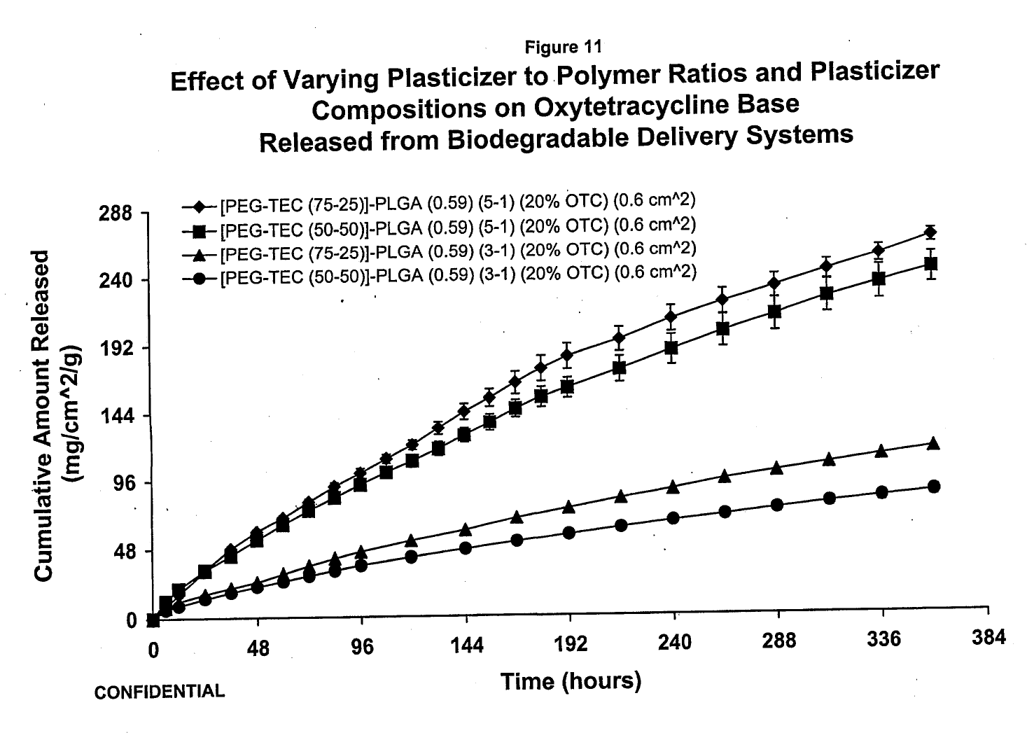

Four different inherent viscosities (i.v.=0.15, 0.26, 0.59 and 0.76) of a polymer (50/50 lactide-co-glycolide copolymer) were dissolved in a minimum quantity of acetone. Pure PEG 400 was added to the polymer solutions. The solutions were stirred to yield uniform mixtures. Acetone was evaporated from the mixtures by heating at 60-75° C. with constant stirring. The blank formulations were kept in a vacuum oven at 60-75° C. overnight to ensure complete removal of acetone. The resulting formulations obtained were matrices with varying viscosities or consistency. The formulation prepared with the polymer of inherent viscosity of 0.76 was considerably more viscous than the one prepared with the polymer of inherent viscosity of 0.15. Oxytetracycline base (20% w/w) was added to each blank formulation and mixed thoroughly to ensure uniform distribution of the drug in the formulations. Drug release from the drug-loaded formulations was performed at 37° C. in isotonic phosphate buffer containing sodium sulfite as an antioxidant. FIG. 11 shows the cumulative amount of oxytetracycline released from formulations prepared with the above-mentioned compositions. It is evident from the figure that decreasing the inherent viscosity of polymer from 0.76 to 0.15 dramatically increased the drug release. This is because a decrease in polymer inherent viscosity resulted in a dramatic decrease in the viscosity of the formulation and a corresponding decease in resistance to drug diffusion from the matrix.

### Example 20

Effect of Varying Drug Solubility on Drug Release

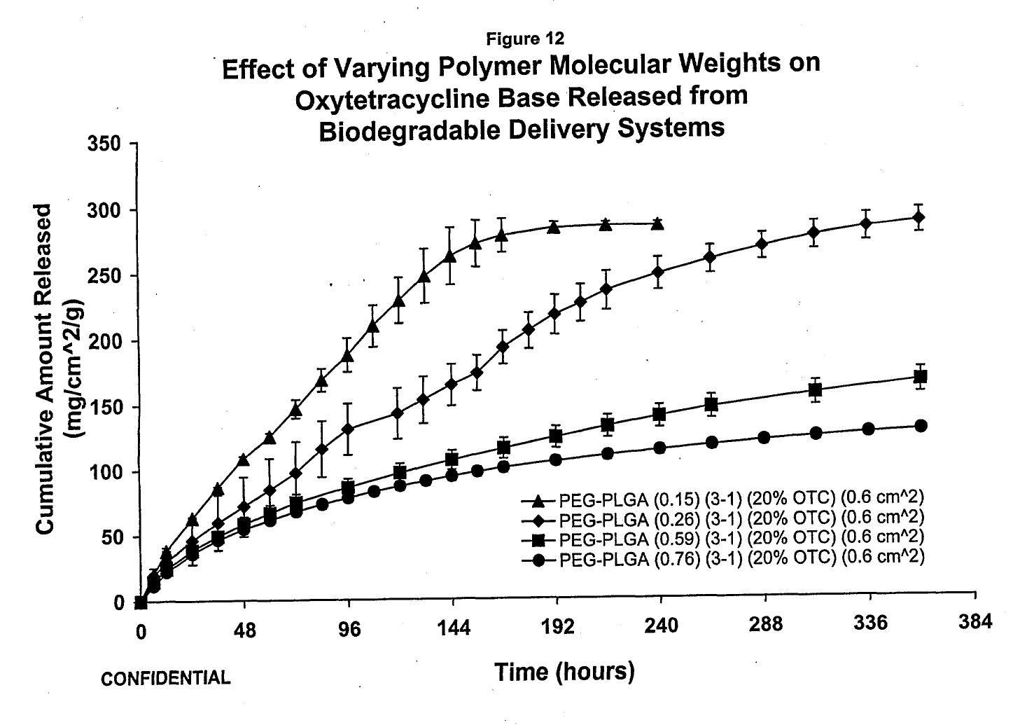

Blank formulations were prepared by dissolving 25% of a polymer (50/50 lactide-co-glycolide copolymer, inherent viscosity of 0.64) and pure PEG 400 or 50/50% blends of PEG 400 and TEC in a minimum quantity of acetone. The solutions were stirred to yield a uniform mixture. Acetone was evaporated from the mixtures by heating at 60-75° C. with constant stirring. The blank formulations were kept in a vacuum oven at 60-75° C. overnight to ensure complete removal of acetone. The resulting formulations obtained were a matrix with viscous liquid-like consistency. Either hydrated naltrexone base (20% w/w) or naltrexone hydrochloride (20% w/w) was added to the blank formulations and mixed thoroughly to ensure uniform distribution of the drugs in the formulations. Drug release from the drug-loaded formulations was performed at 37° C. in isotonic phosphate buffer. FIG. 12 shows the cumulative amount of either hydrated naltrexone base or naltrexone hydrochloride released from formulations prepared with the above-mentioned compositions. The release of naltrexone hydrochloride is considerably faster from formulations prepared with both pure PEG 400 and 50/50% blends of PEG 400 and TEC than the release of the hydrated naltrexone base from similar formulations. This is because the solubility of the naltrexone hydrochloride in the dissolution buffer is much greater than that of the hydrated naltrexone base.

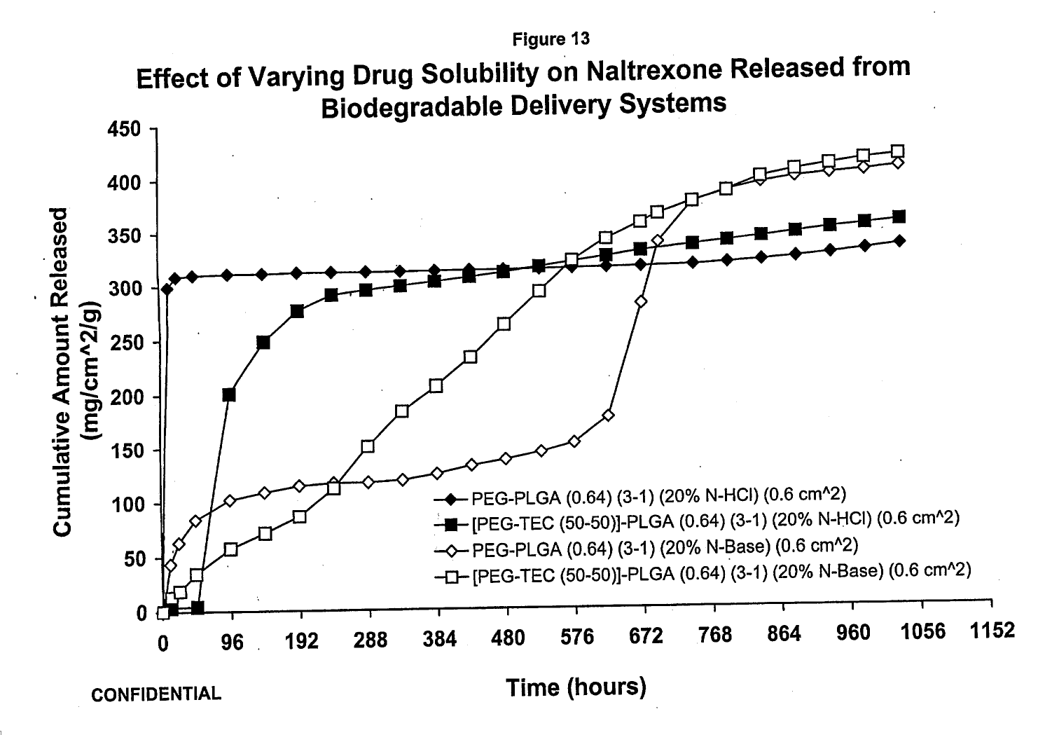

A similar drug release study was performed with formulations containing either 20% oxytetracycline hydrochloride or 20% oxytetracycline base. The blank formulations were prepared by dissolving 25% of a polymer (50/50 lactide-co-glycolide copolymer, inherent viscosity of 0.59) and 75% of pure PEG 400 in a minimum quantity of acetone. The solutions were stirred to yield a uniform mixture. Acetone was evaporated from the mixtures by heating at 60-75° C. with constant stirring. The blank formulations were kept in a vacuum oven at 60-75° C. overnight to ensure complete removal of acetone. The resulting formulations obtained were a matrix with viscous liquid-like consistency. Either 20% oxytetracycline hydrochloride or 20% oxytetracycline base was added to the resulting formulations and mixed thoroughly to ensure uniform drug distribution. Drug release from the drug-loaded formulations was performed at 37° C. in isotonic phosphate buffer containing sodium sulfite as an antioxidant. FIG. 13 shows the cumulative amount of oxytetracycline released from formulations prepared with the above-mentioned compositions. It is evident from the figure that the release of oxytetracycline hydrochloride is considerably faster than the release of oxytetracycline base from similar formulations. This is because of the greater aqueous solubility of the hydrochloride salt than the base.

### Example 21

Biodegradable delivery systems could be prepared by the procedures shown in Examples 1-20. Instead of adding a single biologically active agent, a combination of two or more biologically active agents could be incorporated together in the said delivery system. Examples of some of the combinations of the biologically active agents include levonorgestrel and ethinyl estradiol, trimethoprim and sulfamethoxazole, trimetrexate and leucovorin, isoniazid, rifampin and ethambutol, dapsone and rifampicin, erythromycin and rifampicin, clotrimazole and nystatin, amphotericin B and flucytosine, hydrochlorothiazide and amiloride, hydrochlorothiazide and spironolactone, hydrochlorothiazide and captopril, polythiazide and reserpine. Moreover, instead of adding a single plasticizer, a combination of two or more plasticizers could be added to obtain a formulation with the desired consistency and hydrophilicity or hydrophobicity. An example of a combination of plasticizer is acetyl triacetyl citrate (ATEC), n-methyl pyrrolidone (NMP) and a vegetable oil such as sesame oil, olive oil, safflower oil, sunflower oil, cottonseed oil or almond oil.

### Example 22

Biodegradable vehicle could be prepared by the procedures shown in Examples 1-20. The vehicle could be loaded with BAS in a pharmacy or in an operating room by the health practitioner (a pharmacist, surgeon, nurse), just prior to administration to the patient, with an appropriate quantity of an antitumor agent and injected directly into a solid tumor or at a site from where a solid tumor has been surgically removed. Alternatively, biodegradable vehicle loaded with an antitumor agent can also be injected into the tumor, or injected, implanted, smeared or applied at the site from where the tumor is removed by the surgeon.

### Example 23

A similar treatment described in Example 22 can be offered to patients with brain tumors where the biodegradable vehicle prepared by the methods shown in Examples 1-20 and loaded with an appropriate quantity of an antitumor agent. The BAS-loaded delivery system can be injected, implanted or applied directly at the site in the brain from where the tumor has been removed.

### Example 24

The biodegradable vehicle prepared as shown in examples 1-20 and loaded with a BAS such as an antibiotic an anti-inflammatory agent, a local anesthetic or analgesic, or combinations thereof can also be used in surgeries where appropriate quantities of the BAS, can be mixed with the biodegradable vehicle by the surgeon in an operating room, and the resulting mixture can then be injected, implanted, smeared or applied at the site of surgery to minimize the chances of localized infections or inflammation and reduce pain respectively, due to surgery. Alternatively, an antibiotic loaded biodegradable vehicle can also be injected, implanted, smeared or applied at the site of surgery by the surgeon at the site of surgery.

### Example 25

In the case of orthopedic surgery, a biodegradable vehicle prepared by the method shown in examples 1-20 and loaded with an antibiotic can be injected, implanted, applied or smeared near or at the site of surgery. High concentrations of the antibiotic at the site of surgery can prevent infections. Moreover, the BAS delivery system need not be removed from the site of administration because of the biodegradable nature of the system.

### Example 26

The biodegradable vehicle prepared with the methods described in examples 1-20 and loaded with bone (fragments or powdered) or bone growth promoting agents such as calcium sulfate, calcium phosphates or hydroxyapatite can be injected, implanted, applied or smeared at an appropriate site where it is needed following orthopedic surgery.

### Example 27

The biodegradable vehicle prepared with the methods described in examples 1-20 and loaded with a low molecular weight heparin can also be used to treat conditions such as deep venous thrombosis (DVT) in trauma or surgical patients.

### Example 28

For pulsatile or intermittent delivery of BAS such as vaccines, live or killed viruses or bacteria, the biodegradable vehicle prepared with the methods described in examples 1-20 can be prepared with blends of varying molecular weights of polymers or copolymers, or with blends of copolymers of varying copolymer ratios such as 50/50 PLGA and 85/15 PLGA or 100% polylactic acid (PLA) and 25/75 PLGA, or blends of different types of biodegradable polymers with varying hydrophobicity or lipophilicity or crystallinity such as 1:1 of PLA:PCL or 1:3 of PLA:PCL or 1:1 of 50/50 PLGA:PCL.

### Example 29

The polymer (50/50 lactide-co-glycolide copolymer) was dissolved directly in various plasticizers with stirring with or without the use of heat. Specific examples of formulations prepared using this method are listed in Table 6 below. The resulting formulations obtained were a matrix with a viscous liquid or gel-like consistency.

TABLE 6
Description of formulations prepared by directly mixing the polymer
with the plasticizer with or without the use heat

| | | DESCRIPTION OF THE |
|---|---|---|
| TYPE OF POLYMER | PLASTICIZER | FORMULATION |
| DL-POLYLACTIC ACID | TRIETHYL CITRATE | GEL, TRANSPARENT |
| (DL-PLA; I.V. = 0.58) | (TEC) | GEL, TRANSPARENT |
| DL-POLYLACTIC ACID | ACETYL TRIETHYL CITRATE | GEL, SLIGHTLY CLOUDY |
| (DL-PLA; I.V. = 0.58) | (ATEC) | |
| DL-POLYLACTLC-CO-GLYCOLIC ACID | 2-PYRROLIDONE | LIQUID, |
| (DL-PLGA; I.V. = 0.58) | | TRANSPARENT |
| DL-POLYLACTIC-CO-GLYCOLIC ACID | TRIETHYL CITRATE AND | GEL |
| (DL-PLGA; I.V. = 0.58) | POLYETHYLENE GLYCOL 400 | |
| | (TEC + PEG 400) | |
| DL-POLYLACTIC-CO-GLYCOLIC ACID | ACETYL TRIETHYL CITRATE AND | GEL |
| (DL-PLGA; I.V. = 0.58) | POLYETHYLENE GLYCOL 400 | |
| | (ATEC + PEG 400) | |
| DL-POLYLACTIC-CO-GLYCOLIC ACID | TRIETHYL CITRATE | GEL, SLIGHTLY YELLOW |
| (DL-PLGA; I.V. = 0.58) | (TEC) | |
| DL-POLYLACTIC-CO-GLYCOLIC ACID | N-METHYL PYRROLIDONE | LIQUID, |
| (DL-PLGA; I.V. = 0.58) | (NMP) | TRANSPARENT |
| DL-POLYLACTIC-CO-GLYCOLIC ACID | TRIETHYL CITRATE | VISCOUS LIQUID, |
| (DL-PLGA; I.V. = 0.15) | (TEC) | TRANSPARENT |
| DL-POLYLACTIC-CO-GLYCOLIC ACID | ACETYL TRIETHYL CITRATE | VISCOUS LIQUID, |
| (DL-PLGA; I.V. = 0.15) | (ATEC) | TRANSPARENT |
| DL-POLYLACTIC-CO-GLYCOLIC ACID | TRIETHYL CITRATE | VISCOUS LIQUID, |
| (DL-PLGA; I.V. = 0.15) | (TEC) | TRANSPARENT |
| DL-POLYLACTIC-CO-GLYCOLIC ACID | POLYETHYLENE GLYCOL 400 | VISCOUS LIQUID, |
| (DL-PLGA; I.V. = 0.15) | (PEG − 400) | TRANSPARENT |
| DL-POLYLACTIC-CO-GLYCOLIC ACID | ACETYL TRIETHYL CITRATE AND | LIQUID, |
| (DL-PLGA; I.V. = 0.15) | N-METHYL PYRROLIDONE (NMP) | TRANSPARENT |
| | (ATEC + NMP) | |
| DL-POLYLACTIC-CO-GLYCOLIC ACID | TRIETHYL CITRATE CITRATE AND | LIQUID, |
| (DL-PLGA; I.V. = 0.15) | N-METHYL PYRROLIDONE (NMP) | TRANSPARENT |
| | (TEC + NMP) | |

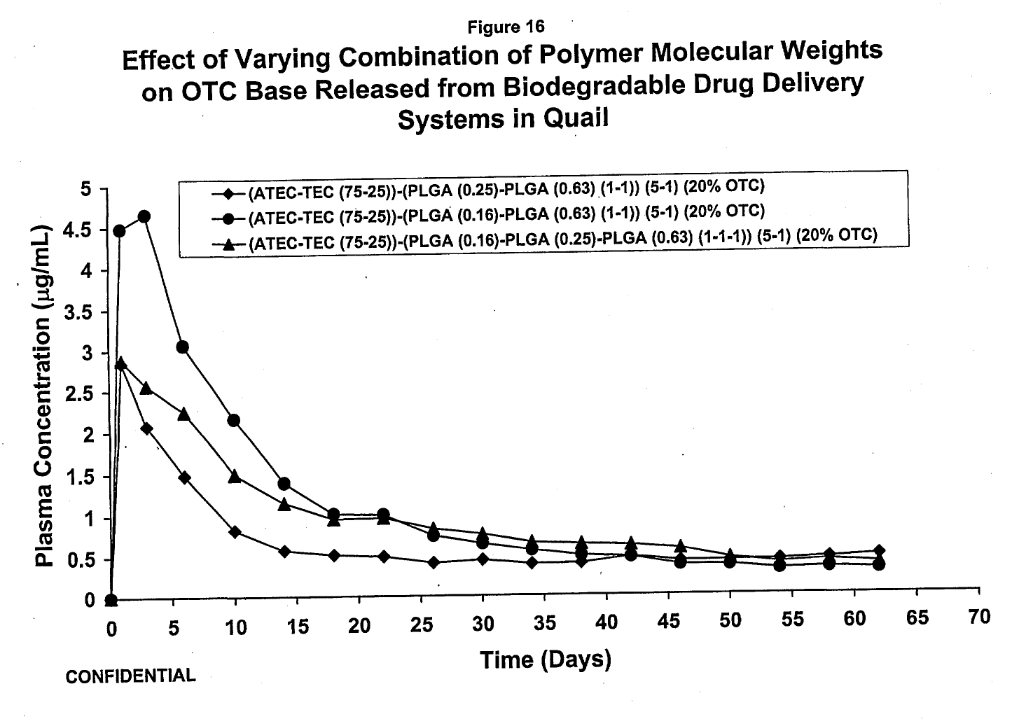

polymer, the desired controlled release of the OTC was achieved in vivo in quail, as seen in FIG. 16.

All publications, patents and patent publications mentioned in this specification are herein incorporated by reference into the specification in their entirety for all purposes. Although the invention has been described with reference to preferred embodiments and examples thereof, the scope of the present invention is not limited only to those described embodiments. As will be apparent to persons skilled in the art, modifications and adaptations to the above-described invention can be made without departing from the spirit and scope of the invention, which is defined and circumscribed by the appended claims.

The foregoing is offered primarily for purposes of illustration. It will be readily apparent to those of ordinary skill in the art that the operating conditions, materials, procedural steps and other parameters of the invention described herein may be further modified or substituted in various ways without departing from the spirit and scope of the invention. For example, the invention has been described with human patients as the usual recipient, but veterinary use is also contemplated. Thus, the preceding description of the invention should not be viewed as limiting but as merely exemplary.

What is claimed is:

1. A biodegradable vehicle or delivery system comprising:
(a) at least one biodegradable polymer; and
(b) at least one plasticizer; said plasticizer being capable of modulating both the consistency and hydrophobicity or hydrophilicity of said biodegradable vehicle or delivery system.

2. The biodegradable delivery system of claim 1 further comprising at least one biologically active substance.

3. The biodegradable vehicle and delivery system of claim 1 wherein said biodegradable polymer is selected from a group consisting of homopolymers and copolymers or blends thereof, of polyesters, polyphosphoesters, polyorthoesters, polylactic acid or polylactides, polyglycolic acid or polyglycolides, polycaprolactones, polyalkylcyanoacrylates, polyphosphazenes, polyhydroxybutyrates, polyhydroxyvalerates, polyaminoacids, pseudopolyamino acids, polyamides, polyanhydrides, polydioxanone, poly(ε-decaloactone), poly(glycolide-co-trimethylene carbonate), poly(ethylene carbonate), poly(iminocarbonate), poly(1,3-propylene malonate), poly(ethylene-1,4-phenylene-bis-oxyacetate), and poly(ester-amides).

4. The biodegradable vehicle and delivery system of claim 1 wherein said plasticizer is selected from a group consisting of citrates such as diethyl citrate (DEC), triethyl citrate (TEC), acetyl triethyl citrate (ATEC), tributyl citrate (TBC), acetyl tributyl citrate (ATBC), butyryltri-n-hexyl-citrate, acetyltri-n-hexyl citrate, phthalates such as dimethyl phthalate (DMP), diethyl phthalate (DEP), dibutyl phthalate (DBP), dioctyl phthalate, glycol ethers such as ethylene glycol diethyl ether, propylene glycol monomethyl ether, ethylene glycol monoethyl ether, diethylene glycol monoethyl ether (Transcutol®), propylene glycol monotertiary butyl ether, dipropylene glycol monomethyl ether, N-methyl-2-pyrrolidone, 2 pyrrolidone (2-Pyrrol®), isopropyl myristate, isopropyl palmitate, dimethylacetamide, propylene glycol, glycerol, glyceryl dioleate, ethyl oleate, benzylbenzoate, glycofurol, sorbitol, sucrose acetate isobutyrate, sebacates such as dibutyl sebacate, dipropylene glycol methyl ether acetate (DPM acetate), propylene carbonate, propylene glycol laurate, propylene glycol caprylate/caprate, gamma butyrolactone, polyethylene glycols (PEG), vegetable oils obtained from seeds, flowers, fruits, leaves, stem or any part of a plant or tree such as cotton seed oil, soy bean oil, almond oil, sunflower oil, peanut oil, sesame oil, glycerol and PEG esters of acids and fatty acids (Gelucires®, Labrafils® and Labrasol®) such as PEG-6 glycerol mono oleate, PEG-6 glycerol linoleate, PEG-8 glycerol linoleate, PEG-4 glyceryl caprylate/caprate, PEG-8 glyceryl caprylate/caprate, polyglyceryl-3-oleate, polyglyceryl-6-dioleate, polyglyceryl-3-isostearate, PEG-32 glyceryl laurate (Gelucire 44/1®), PEG-32 glyceryl palmitostearate (Gelucire 50/13®), PEG-32 glyceryl stearate (Gelucire 53/10®), glyceryl behenate, cetyl palmitate, glyceryl di and tri stearate, glyceryl palmitostearate, and glyceryl triacetate (Triacetin®).

5. The biodegradable delivery system of claim 2 wherein said biologically active substance is selected from the group consisting of steroids, hormones, antipsychotic agents, agents that act on the central nervous system (CNS-agents), narcotic agonists and antagonists, fertility regulating agents, antibodies and antigens, anesthetics, analgesics, antibiotics, antiviral agents, antineoplastic agents, antifungal agents, cavity and infection preventing agents, cardiovascular agents, angiogenic and antiangiogenic agents, anti-inflammatory agents, vasodilators, brochiodilators, alkaloids, peptides and proteins, vaccines, live or killed bacteria and viruses, agents or extracts derived from whole or parts of plants, trees, flowers, fruits, buds, seeds, leaves, barks, stem, roots, and animal tissues, growth promoting agents, soft and hard tissues, growth promoting agents, cells, tissues such as bones or agents derived there from, bone growth promoting agents such as calcium phosphates, calcium sulfate and hydroxyapatites, whole viable cells and cell-lines, deoxyribonucleic acid (DNA), DNA fragments, ribonucleic acid (RNA) RNA fragments, and biological tissues such as islets of langerhans and pancreas. The biologically active substance can be in the form of a solid, or dissolved or suspended in a in a plasticizer or mixtures of plasticizers.

6. A biodegradable vehicle or delivery system comprising:
(a) a combination of two biodegradable polymers, said polymers capable of modulating the degradation kinetics as well as consistency and hydrophobicity or hydrophilicity of the vehicle or delivery system; and
at least one plasticizer, said plasticizer being capable of modulating the consistency and hydrophobicity or hydrophilicity of said biodegradable vehicle or delivery system.

7. A biodegradable vehicle or delivery system comprising:
(a) a combination of three biodegradable polymers, said polymers capable of modulating the degradation kinetics as well as consistency and hydrophobicity or hydrophilicity of the vehicle or delivery system; and
(b) at least one plasticizer, said plasticizer being capable of modulating the consistency and hydrophobicity or hydrophilicity of said biodegradable vehicle or delivery system.

8. (e) A biodegradable vehicle or delivery system comprising:
(a) a combination of two biodegradable polymers, said polymers capable of modulating the degradation kinetics as well as consistency and hydrophobicity or hydrophilicity of the vehicle or delivery system; and
(b) a combination of two plasticizers, said plasticizers being capable of modulating the consistency and hydrophilicity or hydrophobicity of said biodegradable vehicle or delivery system.

9. A biodegradable vehicle or delivery system comprising:
(a) a combination of three biodegradable polymers, said polymers capable of modulating the degradation kinetics as well as consistency and hydrophobicity or hydrophilicity of the vehicle or delivery system; and
(b) a combination of two plasticizers, said plasticizers being capable of modulating the consistency and hydrophilicity or hydrophobicity of said biodegradable vehicle or delivery system.

10. A biodegradable vehicle or delivery system comprising:
(a) at least one biodegradable polymer; and
a combination of two plasticizers, said plasticizers being capable of modulating the consistency and hydrophilicity or hydrophobicity of said biodegradable vehicle or delivery system.

11. A method of preparing a biodegradable vehicle or delivery system comprising the steps of:
(a) selecting at least one biodegradable polymer;
(b) dissolving said polymer in at least one volatile solvent to form a solution;
(c) adding at least one plasticizer to said solution of step (b); and
(d) evaporating said solvent from the solution of step (c).

12. The method of claim 11 wherein said biodegradable polymer is selected from a group consisting homopolymers and copolymers or blends thereof, of polyesters, polyphosphoesters, polyorthoesters, polylactic acid or polylactides, polyglycolic acid or polyglycolides, polycaprolactones, polyalkylcyanoacrylates, polyphosphazenes, polyhydroxybutyrates, polyhydroxyvalerates, polyaminoacids, pseudopolyamino acids, polyamides, polyanhydrides, polydioxanone, poly(ε-decaloactone), poly(glycolide-co-trimethylene carbonate), poly(ethylene carbonate), poly(iminocarbonate), poly(1,3-propylene malonate), poly(ethylene-1,4-phenylene-bis-oxyacetate), and poly(ester-amides).

13. The method of claim 11 wherein said volatile solvent is selected from a group consisting of acetone, methyl acetate, ethyl acetate, chloroform, dichloromethane, methyl ethyl ketone, hexafluroisopropanol, tetrahydrofuran and hexafluroacetone sesquihydrate.

14. The method in claim 11 wherein said plasticizer is selected from a group consisting of citrates such as diethyl citrate (DEC), triethyl citrate (TEC), acetyl triethyl citrate (ATEC), tributyl citrate (TBC), acetyl tributyl citrate (ATBC), butyryltri-n-hexyl-citrate, acetyltri-n-hexyl citrate, phthalates such as dimethyl phthalate (DMP), diethyl phthalate (DEP), dibutyl phthalate (DBP), dioctyl phthalate, glycol ethers such as ethylene glycol diethyl ether, propylene glycol monomethyl ether, ethylene glycol monoethyl ether, diethylene glycol mono ethyl ether (Transcutol®), propylene glycol monotertiary butyl ether, dipropylene glycol monomethyl ether, N-methyl-2-pyrrolidone, 2 pyrrolidone (2-Pyrrol®), isopropyl myristate, isopropyl palmitate, dimethylacetamide, propylene glycol, glycerol, glyceryl dioleate, ethyl oleate, benzylbenzoate, glycofurol, sorbitol, sucrose acetate isobutyrate, sebacates such as dibutyl sebacate, dipropylene glycol methyl ether acetate (DPM acetate), propylene carbonate, propylene glycol laurate, propylene glycol caprylate/caprate, gamma butyrolactone, polyethylene glycols (PEG), vegetable oils obtained from seeds, flowers, fruits, leaves, stem or any part of a plant or tree such as cotton seed oil, soy bean oil, almond oil, sunflower oil, peanut oil, sesame oil, glycerol and PEG esters of acids and fatty acids (Gelucires®, Labrafils® and Labrasol®) such as PEG-6 glycerol mono oleate, PEG-6 glycerol linoleate, PEG-8 glycerol linoleate, PEG-4 glyceryl caprylate/caprate, PEG-8 glyceryl caprylate/caprate, polyglyceryl-3-oleate, polyglyceryl-6-dioleate, polyglyceryl-3-isostearate, PEG-32 glyceryl laurate (Gelucire 44/1®), PEG-32 glyceryl palmitostearate (Gelucire 50/13®), PEG-32 glyceryl stearate (Gelucire 53/10®), glyceryl behenate, cetyl palmitate, glyceryl di and tri stearate, glyceryl palmitostearate, and glyceryl triacetate (Triacetin®).

15. The method in claim 11 further comprising the step of:
(e) adding at least one biologically active substance to the product of step (d) wherein the said biodegradable vehicle is loaded with at least one biologically active substance soon after preparing the biodegradable vehicle or just prior to using the biodegradable delivery system loaded with the biologically active substance.

16. The method in claim 15 wherein said biodegradable polymer is selected from a group consisting homopolymers and copolymers or blends thereof, of polyesters, polyphosphoesters, polyorthoesters, polylactic acid or polylactides, polyglycolic acid or polyglycolides, polycaprolactones, polyalkylcyanoacrylates, polyphosphazenes, polyhydroxybutyrates, polyhydroxyvalerates, polyaminoacids, pseudopolyamino acids, polyamides, polyanhydrides, polydioxanone, poly(ε-decaloactone), poly(glycolide-co-trimethylene carbonate), poly(ethylene carbonate), poly(iminocarbonate), poly(1,3-propylene malonate), poly(ethylene-1,4-phenylene-bis-oxyacetate), and poly(ester-amides).

17. The method of claim 15 wherein said volatile solvent is selected from a group consisting of acetone, methyl acetate, ethyl acetate, chloroform, dichloromethane, methyl ethyl ketone, hexafluroisopropanol, tetrahydrofuran and hexafluroacetone sesquihydrate.

18. The method of claim 15 wherein said plasticizer is selected from a group consisting of citrates such as diethyl citrate (DEC), triethyl citrate (TEC), acetyl triethyl citrate (ATEC), tributyl citrate (TBC), acetyl tributyl citrate (ATBC), butyryltri-n-hexyl-citrate, acetyltri-n-hexyl citrate, phthalates such as dimethyl phthalate (DMP), diethyl phthalate (DEP), dibutyl phthalate (DBP), dioctyl phthalate, glycol ethers such as ethylene glycol diethyl ether, propylene glycol monomethyl ether, ethylene glycol monoethyl ether, diethylene glycol mono ethyl ether (Transcutol®), propylene glycol monotertiary butyl ether, dipropylene glycol monomethyl ether, N-methyl-2-pyrrolidone, 2 pyrrolidone (2-Pyrrol®), isopropyl myristate, isopropyl palmitate, dimethylacetamide, propylene glycol, glycerol, glyceryl dioleate, ethyl oleate, benzylbenzoate, glycofurol, sorbitol, sucrose acetate isobutyrate, sebacates such as dibutyl sebacate, dipropylene glycol methyl ether acetate (DPM acetate), propylene carbonate, propylene glycol laurate, propylene glycol caprylate/caprate, gamma butyrolactone, polyethylene glycols (PEG), vegetable oils obtained from seeds, flowers, fruits, leaves, stem or any part of a plant or tree such as cotton seed oil, soy bean oil, almond oil, sunflower oil, peanut oil, sesame oil, glycerol and PEG esters of acids and fatty acids (Gelucires®, Labrafils® and Labrasol®) such as PEG-6 glycerol mono oleate, PEG-6 glycerol linoleate, PEG-8 glycerol linoleate, PEG-4 glyceryl caprylate/caprate, PEG-8 glyceryl caprylate/caprate, polyglyceryl-3-oleate, polyglyceryl-6-dioleate, polyglyceryl-3-isostearate, PEG-32 glyceryl laurate (Gelucire 44/1®), PEG-32 glyceryl palmitostearate (Gelucire 50/13®), PEG-32 glyceryl stearate (Gelucire 53/10®), glyceryl behenate, cetyl palmitate, glyceryl di and tri stearate, glyceryl palmitostearate, and glyceryl triacetate (Triacetin®).

19. The method of claim 15 wherein said biologically active substance is selected from the group consisting of steroids, hormones, antipsychotic agents, agents that act on the central nervous system (CNS-agents), narcotic agonists and antagonists, fertility regulating agents, antibodies and antigens, anesthetics, analgesics, antibiotics, antiviral agents, antineoplastic agents, antifungal agents, cavity and infection preventing agents, cardiovascular agents, angiogenic and antiangiogenic agents, anti-inflammatory agents, vasodilators, brochiodilators, alkaloids, peptides and proteins, vaccines, live or killed bacteria and viruses, agents or extracts derived from whole or parts of plants, trees, flowers, fruits, buds, seeds, leaves, barks, stem, roots, and animal tissues, growth promoting agents, soft and hard tissues, growth promoting agents, cells, tissues such as bones or agents derived there from, bone growth promoting agents such as calcium phosphates, calcium sulfate and hydroxyapatites, whole viable cells and cell-lines, deoxyribonucleic acid (DNA), DNA fragments, ribonucleic acid (RNA) RNA fragments, and biological tissues such as islets of langerhans and pancreas. The biologically active substance can be in the form of a solid, or dissolved or suspended in a in a plasticizer or mixtures of plasticizers.

20. A method of preparing a biodegradable vehicle or delivery system comprising the steps of:
(a) selecting at least one biodegradable polymer (b) dissolving said polymer in at least one volatile solvent to form a solution;
(c) adding at least one plasticizer to said solution of step (b);
(d) adding at least one biologically active substance to the product of step (c); and
(e) evaporating said solvent from the product of step (d).

21. The method of claim 20 wherein said biodegradable polymer is selected from a group consisting homopolymers and copolymers or blends thereof, of polyesters, polyphosphoesters, polyorthoesters, polylactic acid or polylactides, polyglycolic acid or polyglycolides, polycaprolactones, polyalkylcyanoacrylates, polyphosphazenes, polyhydroxybutyrates, polyhydroxyvalerates, polyaminoacids, pseudopolyamino acids, polyamides, polyanhydrides, polydioxanone, poly(ε-decaloactone), poly(glycolide-co-trimethylene carbonate), poly(ethylene carbonate), poly(iminocarbonate), poly(1,3-propylene malonate), poly(ethylene-1,4-phenylene-bis-oxyacetate), and poly(ester-amides).

22. The method of claim 20 wherein said volatile solvent is selected from a group consisting of acetone, methyl acetate, ethyl acetate, chloroform, dichloromethane, methyl ethyl ketone, hexafluroisopropanol, tetrahydrofuran and hexafluroacetone sesquihydrate.

23. The method of claim 20 wherein said plasticizer is selected from a group consisting of citrates such as diethyl citrate (DEC), triethyl citrate (TEC), acetyl triethyl citrate (ATEC), tributyl citrate (TBC), acetyl tributyl citrate (ATBC), butyryltri-n-hexyl-citrate, acetyltri-n-hexyl citrate, phthalates such as dimethyl phthalate (IMP), diethyl phthalate (DEP), dibutyl phthalate (DBP), dioctyl phthalate, glycol ethers such as ethylene glycol diethyl ether, propylene glycol monomethyl ether, ethylene glycol monoethyl ether, diethylene glycol monoethyl ether (Transcutol®), propylene glycol monotertiary butyl ether, dipropylene glycol monomethyl ether, N-methyl-2-pyrrolidone, 2 pyrrolidone (2-Pyrrol®), isopropyl myristate, isopropyl palmitate, dimethylacetamide, propylene glycol, glycerol, glyceryl dioleate, ethyl oleate, benzylbenzoate, glycofurol, sorbitol, sucrose acetate isobutyrate, sebacates such as dibutyl sebacate, dipropylene glycol methyl ether acetate (DPM acetate), propylene carbonate, propylene glycol laurate, propylene glycol caprylate/caprate, gamma butyrolactone, polyethylene glycols (PEG), vegetable oils obtained from seeds, flowers, fruits, leaves, stem or any part of a plant or tree such as cotton seed oil, soy bean oil, almond oil, sunflower oil, peanut oil, sesame oil, glycerol and PEG esters of acids and fatty acids (Gelucires®, Labrafils® and Labrasol®) such as PEG-6 glycerol mono oleate, PEG-6 glycerol linoleate, PEG-8 glycerol linoleate, PEG-4 glyceryl caprylate/caprate, PEG-8 glyceryl caprylate/caprate, polyglyceryl-3-oleate, polyglyceryl-6-dioleate, polyglyceryl-3-isostearate, PEG-32 glyceryl laurate (Gelucire 44/1®), PEG-32 glyceryl palmitostearate (Gelucire 50/13®), PEG-32 glyceryl stearate (Gelucire 53/10®), glyceryl behenate, cetyl palmitate, glyceryl di and tri stearate, glyceryl palmitostearate, and glyceryl triacetate (Triacetin®).

24. The method of claim 20 wherein said biologically active substance is selected from the group consisting of steroids, hormones, antipsychotic agents, agents that act on the central nervous system (CNS-agents), narcotic agonists and antagonists, fertility regulating agents, antibodies and antigens, anesthetics, analgesics, antibiotics, antiviral agents, antineoplastic agents, antifungal agents, cavity and infection preventing agents, cardiovascular agents, angiogenic and antiangiogenic agents, anti-inflammatory agents, vasodilators, brochiodilators, alkaloids, peptides and proteins, vaccines, live or killed bacteria and viruses, agents or extracts derived from whole or parts of plants, trees, flowers, fruits, buds, seeds, leaves, barks, stem, roots, and animal tissues, growth promoting agents, soft and hard tissues, growth promoting agents, cells, tissues such as bones or agents derived there from, bone growth promoting agents such as calcium phosphates, calcium sulfate and hydroxyapatites, whole viable cells and cell-lines, deoxyribonucleic acid (DNA), DNA fragments, ribonucleic acid (RNA) RNA fragments, and biological tissues such as islets of langerhans and pancreas.

25. A method for modulating the release kinetics of a biologically active substance (BAS) in a biodegradable delivery system comprising a biodegradable polymer, a plasticizer and a BAS, said method comprising:
varying the physiochemical properties of at least one member of the group consisting of said biodegradable polymer, said plasticizer, BAS and combinations thereof, thereby modulating the release kinetics of said biologically active substance (BAS).

26. The method of claim 25, wherein said biologically active substance is selected from the group consisting of steroids, hormones, antipsychotic agents, agents that act on the central nervous system (CNS-agents), narcotic agonists and antagonists, fertility regulating agents, antibodies and antigens, anesthetics, analgesics, antibiotics, antiviral agents, antineoplastic agents, antifungal agents, cavity and infection preventing agents, cardiovascular agents, angiogenic and antiangiogenic agents, anti-inflammatory agents, vasodilators, brochiodilators, alkaloids, peptides and proteins, vaccines, live or killed bacteria and viruses, agents or extracts derived from whole or parts of plants, trees, flowers, fruits, buds, seeds, leaves, barks, stem, roots, and animal tissues, growth promoting agents, soft and hard tissues, growth promoting agents, cells, tissues such as bones or agents derived there from, bone growth promoting agents such as calcium phosphates, calcium sulfate and hydroxyapatites, whole viable cells and cell-lines, deoxyribonucleic acid (DNA), DNA fragments, ribonucleic acid (RNA) RNA fragments, and biological tissues such as islets of langerhans and pancreas.

27. The method of claim 25 wherein said plasticizer is selected from a group consisting of citrates such as diethyl citrate (DEC), triethyl citrate (TEC), acetyl triethyl citrate (ATEC), tributyl citrate (TBC), acetyl tributyl citrate (ATBC), butyryltri-n-hexyl-citrate, acetyltri-n-hexyl citrate, phthalates such as dimethyl phthalate (DMP), diethyl phthalate (DEP), dibutyl phthalate (DBP), dioctyl phthalate, glycol ethers such as ethylene glycol diethyl ether, propylene glycol monomethyl ether, ethylene glycol monoethyl ether, diethylene glycol monoethyl ether (Transcutol®), propylene glycol monotertiary butyl ether, dipropylene glycol monomethyl ether, N-methyl-2-pyrrolidone, 2 pyrrolidone (2-Pyrrol®), isopropyl myristate, isopropyl palmitate, dimethylacetamide, propylene glycol, glycerol, glyceryl dioleate, ethyl oleate, benzylbenzoate, glycofurol, sorbitol, sucrose acetate isobutyrate, sebacates such as dibutyl sebacate, dipropylene glycol methyl ether acetate (DPM acetate), propylene carbonate, propylene glycol laurate, propylene glycol caprylate/caprate, gamma butyrolactone, polyethylene glycols (PEG), vegetable oils obtained from seeds, flowers, fruits, leaves, stem or any part of a plant or tree such as cotton seed oil, soy bean oil, almond oil, sunflower oil, peanut oil, sesame oil, glycerol and PEG esters of acids and fatty acids (Gelucires®, Labrafils® and Labrasol®) such as PEG-6 glycerol mono oleate, PEG-6 glycerol linoleate, PEG-8 glycerol linoleate, PEG-4 glyceryl caprylate/caprate, PEG-8 glyceryl caprylate/caprate, polyglyceryl-3-oleate, polyglyceryl-6-dioleate, polyglyceryl-3-isostearate, PEG-32 glyceryl laurate (Gelucire 44/1®), PEG-32 glyceryl palmitostearate (Gelucire 50/13®), PEG-32 glyceryl stearate (Gelucire 53/10®), glyceryl behenate, cetyl palmitate, glyceryl di and tri stearate, glyceryl palmitostearate, and glyceryl triacetate (Triacetin®).

28. The method of claim 25 wherein said biodegradable polymer is selected from a group consisting homopolymers and copolymers or blends thereof, of polyesters, polyphosphoesters, polyorthoesters, polylactic acid or polylactides, polyglycolic acid or polyglycolides, polycaprolactones, polyalkylcyanoacrylates, polyphosphazenes, polyhydroxybutyrates, polyhydroxyvalerates, polyaminoacids, pseudopolyamino acids, polyamides, polyanhydrides, polydioxanone, poly(ε-decaloactone), poly(glycolide-co-trimethylene carbonate), poly(ethylene carbonate), poly(iminocarbonate), poly(1,3-propylene malonate), poly(ethylene-1,4-phenylene-bis-oxyacetate), and poly(ester-amides).

29. A method for modulating the degradation kinetics of a biodegradable delivery vehicle comprising a biodegradable polymer, a plasticizer and optionally a biologically active substance said method comprising:
varying the physiochemical properties of at least one member of the group consisting of said biodegradable polymer, said plasticizer, optionally a BAS and combinations thereof, thereby modulating the degradation kinetics of said biodegradable vehicle.
